SEQUENCE LISTING (49 sequences)

SEQ ID NO: 1 - DNA, 53 bp, synthetic construct
  source 1..53: mol_type = other DNA
tcgtcggcagcgtcagatgtgtataagagacagvbhdvccagtctggccaggg

SEQ ID NO: 2 - DNA, 52 bp, synthetic construct
  source 1..52: mol_type = other DNA
gtctcgtgggctcggagatgtgtataagagacaggtgatgccgtagtactgg

SEQ ID NO: 3 - protein, 8 aa, synthetic construct
  VARIANT 7: note = X can be any amino acid
  VARIANT 8: note = X can be E or D
  VARIANT 3..4: note = X can be any amino acid
QPXXPFXX

SEQ ID NO: 4 - skipped

SEQ ID NO: 5 - protein, 7 aa, synthetic construct
  VARIANT 2: note = X can be any amino acid
  VARIANT 6: note = X can be any amino acid
KXGHDXC

SEQ ID NO: 6 - protein, 7 aa, synthetic construct
  VARIANT 6: note = X can be any amino acid
  VARIANT 7: note = X can be I or V
  VARIANT 4: note = X can be any amino acid
KGGXDXX

SEQ ID NO: 7 - protein, 6 aa, synthetic construct
  VARIANT 2: note = X can be any amino acid
  VARIANT 5: note = X can be I or V
KXHGXF

SEQ ID NO: 8 - protein, 6 aa, synthetic construct
  VARIANT 5: note = X can be any amino acid
HWFDXW

SEQ ID NO: 9 - skipped

SEQ ID NO: 10 - protein, 5 aa, synthetic construct
ILQAD

SEQ ID NO: 11 - protein, 7 aa, synthetic construct
  VARIANT 5..6: note = X can be any amino acid
LHADXXF

SEQ ID NO: 12 - protein, 5 aa, synthetic construct
  VARIANT 1: note = X can be I, V, T or P
XLHAD

SEQ ID NO: 13 - protein, 6 aa, synthetic construct
  VARIANT 3: note = X can be any amino acid
  VARIANT 6: note = X can be R, S or Q
TLXADX

SEQ ID NO: 14 - protein, 6 aa, synthetic construct
  VARIANT 2: note = X can be any amino acid
KXIPAE

SEQ ID NO: 15 - protein, 6 aa, synthetic construct
  VARIANT 1: note = X can be E or D
  VARIANT 2: note = X can be any amino acid
XXAGYN

SEQ ID NO: 16 - skipped

SEQ ID NO: 17 - protein, 6 aa, synthetic construct
  VARIANT 2: note = X can be R or L
  VARIANT 3: note = X can be any amino acid
CXXTDC

SEQ ID NO: 18 - skipped

SEQ ID NO: 19 - protein, 5 aa, synthetic construct
LPADR

SEQ ID NO: 20 - protein, 7 aa, synthetic construct
  VARIANT 1: note = X can be M or L
  VARIANT 2..3: note = X can be any amino acid
XXXLSAD

SEQ ID NO: 21-22 - skipped (2 entries)

SEQ ID NO: 23 - protein, 6 aa, synthetic construct
  VARIANT 1: note = X can be F, Y or L
  VARIANT 2: note = X can be any amino acid
XXSAQP

SEQ ID NO: 24-25 - skipped (2 entries)

SEQ ID NO: 26 - protein, 5 aa, synthetic construct
  VARIANT 5: note = X can be E or D
MAGMX

SEQ ID NO: 27 - protein, 5 aa, synthetic construct
CMAGM

SEQ ID NO: 28 - protein, 5 aa, synthetic construct
CMAGE

SEQ ID NO: 29 - protein, 5 aa, synthetic construct
GPAGZ

SEQ ID NO: 30 - protein, 5 aa, synthetic construct
  VARIANT 5: note = X can be T or D
PAAGX

SEQ ID NO: 31-33 - skipped (3 entries)

SEQ ID NO: 34 - protein, 6 aa, synthetic construct
  VARIANT 2: note = X can be any amino acid
CXDPQC

SEQ ID NO: 35 - protein, 5 aa, synthetic construct
  VARIANT 1: note = X can be L or F
XEDKS

SEQ ID NO: 36 - protein, 6 aa, synthetic construct
  VARIANT 2: note = X can be any amino acid
SXNLEQ

SEQ ID NO: 37 - protein, 12 aa, synthetic construct
EAKILHADLTEK

SEQ ID NO: 38 - protein, 12 aa, synthetic construct
SNPHWFDTWIRV

SEQ ID NO: 39 - protein, 12 aa, synthetic construct
APRKAIPAENRL

SEQ ID NO: 40 - protein, 12 aa, synthetic construct
TKADIAGYNIVD

SEQ ID NO: 41 - protein, 12 aa, synthetic construct
NIKGGYDILTIA

SEQ ID NO: 42 - protein, 12 aa, synthetic construct
HQGWAAGTIVVG

SEQ ID NO: 43 - protein, 12 aa, synthetic construct
VWGLGPAGQWDT

SEQ ID NO: 44 - protein, 12 aa, synthetic construct
KWPGIFSMPPMD

SEQ ID NO: 45 - protein, 12 aa, synthetic construct
RSQKQQGIFSFA

SEQ ID NO: 46 - protein, 12 aa, synthetic construct
KHFGIFSEDSWQ

SEQ ID NO: 47 - protein, 12 aa, synthetic construct
HYTEYFNYDPRK

SEQ ID NO: 48 - protein, 11 aa, synthetic construct
GGRWQYTEDFN

SEQ ID NO: 49 - protein, 12 aa, synthetic construct
ALQDWHFTEQYN

(19) United States
(12) Patent Application Publication
(10) Pub. No.: US 2024/0192208 A1
(43) Pub. Date: Jun. 13, 2024

(54) SERUM ANTIBODY PROFILING FOR LEPTOSPIROSIS

(71) Applicant: Serimmune Inc., Goleta, CA (US)

(72) Inventor: Kathryn Vinaya Louise Kamath, Santa Barbara, CA (US)

(21) Appl. No.: 18/507,763

(22) Filed: Nov. 13, 2023

Related U.S. Application Data
(63) Continuation of application No. PCT/US2022/029258, filed on May 13, 2022.
(60) Provisional application No. 63/188,430, filed on May 13, 2021.

(51) Int. Cl. G01N 33/569

(52) U.S. Cl. CPC G01N 33/56911; G01N 2333/20; G01N 2469/20

(57) ABSTRACT

Disclosed herein are reagents for use in antibody profiling platforms, such as biopanning and Digital Serology, specifically for eptiope motifs directed to Leptospira. Also disclosed herein are kits, method of manufacturing, and methods of using the same.

### 1. CROSS REFERENCE TO RELATED APPLICATIONS

This application is a continuation of PCT/US2022/029258, filed May 13, 2022, which claims the benefit of U.S. Provisional Application No. 63/188,430, filed May 13, 2021, each of which is hereby incorporated in their entirety by reference for all purposes.

### 2. SEQUENCE LISTING

The instant application contains a Sequence Listing which has been submitted via Patent Center and is hereby incorporated by reference in its entirety. Said XML copy, created on Dec. 26, 2023, is named SUI-008WOC1, and is 47,130 bytes in size.

### 3. BACKGROUND

Serology is the practice of identifying components of serum samples. In particular, several technologies exist in the field for identifying antibodies present in serum. For example, biopanning using peptide displays is a technique used to enrich for antibodies specific to particular protein targets. Biopanning assays have also been applied to epitope identification.

Leptospirosis is a potentially serious fatal infection caused by spirochetes of the genus Leptospira. There are more than 250 antigenically distinct serovars. The Microscopic agglutination test (MAT) test is the most widely used reference test and involves titrating serum on slide prepared with a single live Leptospires serovar. A patient suspected of Leptospirosis is tested with serovars endemic to the specific region. A four-fold change in the antibody titer between paired acute and convalescent samples suggests current Leptospira infection. Reported sensitivity for MAT testing is as low (˜55%) (Niloofa et al. Diagnosis of Leptospirosis: Comparison between Microscopic Agglutination Test, IgM-ELISA and IgM Rapid Immunochromatography Test. PLOS One. 2015 Jun. 18; 10(6):e0129236; Chirathaworn et al. Interpretation of microscopic agglutination test for leptospirosis diagnosis and seroprevalence. Asian Pac J Trop Biomed. 2014 May; 4(Suppl 1):S162-4; Alia et al. Diagnostic accuracy of rapid diagnostic tests for the early detection of leptospirosis. J Infect Public Health. 2019 March-April; 12(2):263-269), particularly in the acute phase of infection. Other diagnostic tests including, PCR, IFA, ELISA, Immunochromatography and Lateral flow have similarly low sensitivities (≤85%) and higher sensitivity tests that come at the expense of specificity (Niloofa et al. Diagnosis of Leptospirosis: Comparison between Microscopic Agglutination Test, IgM-ELISA and IgM Rapid Immunochromatography Test. PLOS One. 2015 Jun. 18; 10(6):e0129236)

Absent from the field are standardized Leptospirosis diagnostic tests with improved sensitivity and specificity, particularly during the acute phase of infection when intervention is most critical.

### 4. SUMMARY

Serum Epitope Repertoire Analysis (SERA) utilizes bacterial display peptide library technology coupled with next-generation sequencing to profile antibody repertoires and identify IgM and IgG antigenic epitopes, in the form of peptide motifs, that are diagnostic for Leptospirosis infection. The motifs identified are enriched and highly specific to Leptospirosis patients relative to endemic and non-endemic controls and some of the motifs map to known Leptospiral antigens. When combined into a panel, the motifs yield a diagnostic test with superior sensitivity and specificity relative to gold standard MAT testing.

Accordingly, the invention described herein provides compositions and methods directed to epitopes associated with Leptospirosis.

Provide herein is a peptide display system, wherein the display system comprises: (a) one or more display surfaces; and (b) at least one distinct peptide, wherein the at least one distinct peptides comprises a binding motif, wherein the binding motif is selected from a motif group consisting of: [FM]TEX[FY]N (SEQ ID NO:4), KXGHDXC (SEQ ID NO:5), KGGXDX[IV] (SEQ ID NO:6), KXHG[IV]F (SEQ ID NO:7), HWFDXW (SEQ ID NO:8), HW[FL]DX[WF] (SEQ ID NO:9), ILQAD (SEQ ID NO:10), LHADXXF (SEQ ID NO:11), [IVTP]LHAD (SEQ ID NO:12), TLXAD[RSQ] (SEQ ID NO:13), KXIPAE (SEQ ID NO:14), [ED]XAGYN (SEQ ID NO:15), [EDPA]X[AG][GA]YN (SEQ ID NO:16), C[RL]XTDC (SEQ ID NO:17), [LIV]L[QH]AE (SEQ ID NO:18), LPADR (SEQ ID NO:19), [ML]XXLSAD (SEQ ID NO:20), [FWY]XP[RV]AD (SEQ ID NO:21), AD[QR]E[HQ] (SEQ ID NO:22), [FYL]XSAQP (SEQ ID NO:23), [SD]AQPX[WF] (SEQ ID NO:24), [TS]AQPXX[WR] (SEQ ID NO:25), MAGM[ED] (SEQ ID NO:26), CMAGM (SEQ ID NO:27), CMAGE (SEQ ID NO:28), GPAG[EQ] (SEQ ID NO:29), PAAG[TD] (SEQ ID NO:30), PAG[TD]XXX[WRSHTG] (SEQ ID NO:31), PA[GA][TD]XW (SEQ ID NO:32), [AP]AGT[MLI] (SEQ ID NO:33), CXDPQC (SEQ ID NO:34), [LF]EDKS (SEQ ID NO:35), and SXNLEQ (SEQ ID NO:36), and wherein the at least one distinct peptide extends from the one or more display surfaces.

In some aspects, the at least one distinct peptide comprising the binding motif LHADXXF (SEQ ID NO:11) comprises the amino acid sequence EAKILHADLTEK (SEQ ID NO:37). In some aspects, the at least one distinct peptide comprising the binding motif HWFDXW (SEQ ID NO:8) comprises the amino acid sequence selected from the group consisting of: SNPHWFDTWIRV (SEQ ID NO:38), APRKAIPAENRL (SEQ ID NO:39), TKADIAGYNIVD (SEQ ID NO:40), NIKGGYDILTIA (SEQ ID NO:41), and combinations thereof. In some aspects, the at least one distinct peptide comprising the binding motif selected from the group consisting of: MAGM[ED] (SEQ ID NO:26), CMAGM (SEQ ID NO:27), CMAGE (SEQ ID NO:28), GPAG[EQ] (SEQ ID NO:29): PA[GA][TD]XW (SEQ ID NO:32); PAAG[TD] (SEQ ID NO:30): [AP]AGT[MLI] (SEQ ID NO:33) comprises the amino acid sequence HQGWAAGTIVVG (SEQ ID NO:42) and/or VWGLGPAGQWDT (SEQ ID NO:43).

In some aspects, the at least one distinct peptide comprising the binding motif KXHG[IV]F (SEQ ID NO:7) comprises the amino acid sequence selected from the group consisting of: KWPGIFSMPPMD (SEQ ID NO:44), RSQKQQGIFSFA (SEQ ID NO:45), KHFGIFSEDSWQ (SEQ ID NO:46), and combinations thereof. In some aspects, the at least one distinct peptide comprising the binding motif [FM]TEX[FY]N (SEQ ID NO:4) comprises the amino acid sequence selected from the group consisting of: HYTEYFNYDPRK (SEQ ID NO:47), GGRWQYTEDEN (SEQ ID NO:48), ALQDWHFTEQYN (SEQ ID NO:49), and combinations thereof.

In some aspects, the display system comprises at least 2 distinct peptides each comprising a distinct binding motif, wherein each distinct binding motif is selected from the motif group. In some aspects, the display system comprises at least 3, at least 4, or at least 5 distinct peptides each comprising a distinct binding motif, wherein each distinct binding motif is selected from the motif group. In some aspects, the display system comprises at least 6, at least 7, at least 8, or at least 9 distinct peptides each comprising a distinct binding motif, wherein each distinct binding motif is selected from the motif group. In some aspects, the display system comprises at least 10, at least 15, at least 20, at least 25, or at least 30 distinct peptides each comprising a distinct binding motif, wherein each distinct binding motif is selected from the motif group.

In some aspects, the at least one distinct peptide is capable of binding to an antibody associated with a Leptospirosis infection.

In some aspects, the one or more display surfaces comprises a single display surface. In some aspects, the single display surface comprises a solid support. In some aspects, each of the at least one distinct peptides is attached at a defined location on the single display surface. In some aspects, the single display surface is a peptide microarray. In some aspects, the defined location on the peptide microarray is a defined grid location on the peptide microarray. In some aspects, the single display surface is a multi-well plate. In some aspects, the defined location in the multi-well plate is a defined well in the multi-well plate. In some aspects, the single display surface comprises a lateral flow assay. In some aspects, the defined location in the lateral flow assay is a defined location on a capillary membrane.

In some aspects, the one or more display surfaces comprises a plurality of display surfaces. In some aspects, the plurality of display surfaces is a plurality of biological entity surfaces. In some aspects, the biological entity comprises a cell, a virus, or a bacteriophage. In some aspects, the at least one distinct peptide is encoded by one or more distinct nucleic acid sequences capable of expressing the at least one distinct peptide in the plurality of biological entities. In some aspects, the at least one distinct peptide comprises a surface display peptide sequence configured to be expressed as part of the peptide and capable of directing the peptides for display on the biological entity surface. In some aspects, the biological entity is an Escherichia coli cell. In some aspects, the at least one distinct peptide comprises at least a portion of an Escherichia coli eCPX scaffold. In some aspects, the plurality of display surfaces is a plurality of microparticle surfaces. In some aspects, each of the plurality of display surfaces comprises a distinct single species of the at least one distinct peptides.

In some aspects, the at least one distinct peptide further comprises a label.

In some aspects, the one or more display surfaces or the distinct peptides is bound or capable of being bound directly or indirectly to a capture entity. In some aspects, the capture entity comprises a solid support selected from the group consisting of: an agarose bead, a sepharose bead, a magnetic bead, and a resin. In some aspects, the capture entity comprises a binding moiety that binds directly to the one or more display surfaces or the distinct peptides. In some aspects, the capture entity comprises a binding moiety that binds a binding molecule that binds to the one or more display surfaces or at least one of the distinct peptides. In some aspects, the binding molecule that binds to at least one of the distinct peptides is an antibody from a biological sample. In some aspects, the binding moiety is selected from the group consisting of: Protein A, Protein G, Protein A/G, and an antibody or antigen binding fragment thereof. In some aspects, the antibody or antigen binding fragment thereof comprises an anti-immunoglobulin antibody. In some aspects, the binding moiety comprises streptavidin and the one or more display surfaces, the distinct peptide, or the binding molecule that binds to the one or more display surfaces or at least one of the distinct peptides further comprises biotin. In some aspects, the capture entity further comprises a fluorophore.

In some aspects, displaying comprises presentation of each peptide for direct binding by a binding molecule. In some aspects, the binding molecule is selected from the group consisting of: an antibody or antigen-binding fragment thereof, an aptamer, a peptoid, and an affibody. In some aspects, the binding molecule is an antibody or antigen-binding fragment thereof.

In some aspects, the composition further comprises a library of peptides. In some aspects, the library of peptides is a random peptide library. In some aspects, the library of peptides is a non-random peptide library. In some aspects, the non-random library is biased to represent one or more diseases or conditions, and wherein at least one of the one or more diseases or conditions is a Leptospirosis infection. In some aspects, the biased representation is at least greater than a representation in a random peptide library. In some aspects, the greater representation is statistically significant, wherein the statistical significance has a p-value less than 0.05. In some aspects, each of the peptides in the library of peptides is at least 5, 6, 7, 8, 9, 10, 11, 12, 13, 14, 15, 16, 17, 18, 19, or 20 amino acids in length.

In some aspects, each of the distinct peptides and each of the peptides in the library of peptides is an identical defined length. In some aspects, the identical defined length is 5, 6, 7, 8, 9, 10, 11, 12, 13, 14, 15, 16, 17, 18, 19, or 20 amino acids in length. In some aspects, the identical defined length is 12 amino acids in length.

In some aspects, two or more of the at least one distinct peptides or two or more of the library of peptides are concatenated. In some aspects, each of the at least one distinct peptides or each of the library of peptides are concatenated. In some aspects, each of the at least one distinct peptides is concatenated in a single polypeptide.

In some aspects, the diversity of the library is less than 8×10⁹ distinct peptides. In some aspects, the diversity of the library is 384 or fewer, 96 or fewer, 48 or fewer, 24 or fewer, or 12 or fewer distinct peptides. In some aspects, the diversity of the library is at least 10, at least 100, at least 1000, at least 10⁴, at least 10⁵, at least 10⁶, at least 10⁷, at least 10⁸ distinct peptides.

In some aspects, the binding motifs are selected from IgG isotype specific binding motifs. In some aspects, the binding motifs are selected from IgM isotype specific binding motifs.

Also provided herein is a method of determining the presence of specimen antibodies specific for Leptospirosis in a biological sample comprising: contacting any one of the display systems described herein with a biological sample, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope; measuring the binding between the at least one distinct peptide and the specimen antibodies; and determining specimen antibodies specific for Leptospirosis are present in the biological sample when binding between at least one of the at least one distinct peptides and the specimen antibodies is detected.

Also provided herein is a method of treating a subject known or suspected of suffering from a Leptospirosis infection comprising assessing or having assessed whether the subject suffers from a Leptospirosis infection, wherein the assessment comprises: contacting any one of the display systems described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope; measuring the binding between the at least one distinct peptide and the specimen antibodies; and assessing the subject as: i) positive for the Leptospirosis infection, or ii) negative for the Leptospirosis infection; and administering or having administered a therapy to treat the Leptospirosis infection when the subject when the subject is assessed as positive.

Also provided herein is a method of determining whether a subject is a candidate for treatment of a Leptospirosis infection comprising assessing or having assessed whether the subject suffers from a Leptospirosis infection, wherein the assessment comprises: contacting any one of the display systems described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope; measuring the binding between the at least one distinct peptide and the specimen antibodies; and diagnosing the subject as: i) positive for the Leptospirosis infection, or ii) negative for the Leptospirosis infection, determining the subject is a candidate for a treatment of the Leptospirosis infection when the subject is assessed as positive.

Also provided herein is a method of diagnosing a Leptospirosis infection in a subject comprising: contacting any one of the display systems described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope; measuring the binding between the at least one distinct peptide and the specimen antibodies; and diagnosing the subject as: i) positive for the Leptospirosis infection, or ii) negative for the Leptospirosis infection.

In some aspects, the assessing or diagnosing comprises assessing or diagnosing the subject as: i) positive for the Leptospirosis infection when binding between at least one of the at least one distinct peptides and the specimen antibodies is detected, or ii) negative for the Leptospirosis infection when binding between less than at least one of the at least one distinct peptides and the specimen antibodies is detected.

In some aspects, the assessing or diagnosing comprises assessing or diagnosing the subject as: i) positive for the Leptospirosis infection when binding between at least 2, at least 3, at least 4, or at least 5 distinct peptides of the at least one distinct peptides and the specimen antibodies is detected, or ii) negative for the Leptospirosis infection when binding between less than at least 2, at least 3, at least 4, or at least 5 distinct peptides, respectively, of the at least one distinct peptides and the specimen antibodies is detected.

In some aspects, the assessing or diagnosing comprises assessing or diagnosing the subject as: i) positive for the Leptospirosis infection when binding between at least 6, at least 7, at least 8, at least 9, or at least 10 distinct peptides of the at least one distinct peptides and the specimen antibodies is detected, or ii) negative for the Leptospirosis infection when binding between less than at least 6, at least 7, at least 8, at least 9, or at least 10 distinct peptides, respectively, of the at least one distinct peptides and the specimen antibodies is detected.

In some aspects, the measuring comprises calculating a z-score for binding between the at least one distinct peptide and the specimen antibodies and the assessing or diagnosing comprises assessing or diagnosing the subject as: i) positive for the Leptospirosis infection when the z-score meets or exceeds a threshold value, or ii) negative for the Leptospirosis infection when the z-score is less than the threshold value.

In some aspects, the method further comprises contacting the biological sample with at least one reagent configured to remove antibodies that bind to array components other than the at least one distinct peptide before contacting the display system of with the biological sample from the subject.

In some aspects, the at least one reagent comprises the one or more display surfaces free of the at least one distinct peptide.

In some aspects, the measuring comprises use of an assay selected from the group consisting of: optical scanning, an ELISA, fluorescent detection, digital serology, flow-cytometry, surface plasmon resonance, bio-layer interferometry, lateral flow assay, or combinations thereof.

In some aspects, the method further comprises determining amino acid sequences of peptides in the display system bound by the specimen antibodies.

In some aspects, the method further comprises determining binding motifs of peptides in the display system bound by the specimen antibodies using a clustering algorithm.

In some aspects, the method further comprises determining the isotype of the specimen antibodies that bind to the peptides in the display system. In some aspects, the method further comprises determining the isotype of the specimen antibodies that bind to the at least one distinct peptide.

In some aspects, the method further comprises prior to the measuring step isolating specific specimen antibody isotypes. In some aspects, the measuring step further comprises measuring the binding between the at least one distinct peptide and specific specimen antibody isotypes. In some aspects, the isotype is IgM. In some aspects, the isotype is IgG or subclasses thereof.

In some aspects, calculating the z-score comprises calculating a composite z-score for binding between each of the at least one distinct peptides specific for Leptospirosis and the specimen antibodies.

In some aspects, the subject is known or suspected of having an acute Leptospirosis infection. In some aspects, the subject is known or suspected of having a convalescent Leptospirosis infection. In some aspects, the subject was previously diagnosed as having a Leptospirosis infection.

In some aspects, the biological sample comprises a bodily fluid. In some aspects, the bodily fluid is peripheral blood, lymphatic fluid, sweat, saliva, or mucus.

In some aspects, the method has a sensitivity of at least 60%, at least 70%, at least 80%, at least 90%, or at least 95%. In some aspects, the method has a sensitivity of at least 80%. In some aspects, the method has a sensitivity of at least 90%. In some aspects, the method has a sensitivity of at least 94%. In some aspects, the method has a sensitivity of at least 97%. In some aspects, the method has a sensitivity of 100%. In some aspects, the method has a sensitivity of at least 91%, at least 92%, at least 93%, at least 94%, at least 95%, at least 96%, at least 97%, at least 98%, at least 99%, or 100%. In some aspects, the method has a sensitivity of 100%.

In some aspects, the biological sample is from a subject suspected of having an acute Leptospirosis infection, and wherein the method has a sensitivity of at least 60%, optionally wherein the binding motifs are selected from IgG isotype specific binding motifs. In some aspects, the biological sample is from a subject suspected of having an acute Leptospirosis infection, and wherein the method has a sensitivity of at least 80%, optionally wherein the binding motifs are selected from both IgG and IgM isotype specific binding motifs. In some aspects, the biological sample is from a subject suspected of having an acute Leptospirosis infection, and wherein the method has a sensitivity of at least 90%, optionally wherein the binding motifs are selected from IgM isotype specific binding motifs. In some aspects, the biological sample is from a subject suspected of having an acute Leptospirosis infection, and wherein the method has a sensitivity of at least 90%, optionally wherein the binding motifs are selected from both IgG and IgM isotype specific binding motifs.

In some aspects, the method has a sensitivity greater than a microscopic agglutination test. In some aspects, the method has a sensitivity of at least 60%, at least 61%, at least 62%, at least 63%, at least 64%, at least 65%, at least 66%, at least 67%, at least 68%, at least 69%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, or at least 89%.

In some aspects, the method has a specificity greater than a microscopic agglutination test. In some aspects, the method has a specificity of at least 90%, at least 91%, at least 92%, at least 93%, at least 94%, at least 95%, at least 96%, at least 97%, at least 98%, or at least 99%. In some aspects, the method has a specificity of at least 96%. In some aspects, the method has a specificity of at least 97%. In some aspects, the method has a specificity of at least 99%. In some aspects, the binding motifs are selected from both IgG and IgM isotype specific binding motifs, and wherein the method has a specificity of at least 96%. In some aspects, the binding motifs are selected from IgM isotype specific binding motifs, and wherein the method has a specificity of at least 97%. In some aspects, the binding motifs are selected from IgG isotype specific binding motifs, and wherein the method has a specificity of at least 99%. In some aspects, the binding motifs are selected from IgG isotype specific binding motifs, and wherein the method has a specificity of at least 99.5%.

In some aspects, the method has a sensitivity of at least 60% and a specificity of at least 90%. In some aspects, the method has a sensitivity of at least 60% and a specificity of at least 95%. In some aspects, the method has a sensitivity of at least 80% and a specificity of at least 90%. In some aspects, the method has a sensitivity of at least 80% and a specificity of at least 95%. In some aspects, the method has a sensitivity of at least 90% and a specificity of at least 90%. In some aspects, the method has a sensitivity of at least 90% and a specificity of at least 95%.

Also provided for herein is a kit for diagnosing a Leptospirosis infection, comprising the display system of any of the compositions described herein and a system for detecting the binding of each of the at least one distinct peptides to its target antibody. In some aspects, the kit further comprises an agent for removing antibodies that bind to array components other than the at least one distinct peptide.

Also provided for herein is a peptide expression library, wherein the library comprises a library of nucleic acid sequences encoding a library of peptides, wherein the library of peptides comprises at least one distinct peptide, wherein the at least one distinct peptide comprises a binding motif, wherein the binding motif is selected from a motif group consisting of:

(SEQ ID NO: 4)
[FM]TEX[FY]N,
(SEQ ID NO: 5)
KXGHDXC,
(SEQ ID NO: 6)
KGGXDX[IV],
(SEQ ID NO: 7)
KXHG[IV]F,
(SEQ ID NO: 8)
HWFDXW,
(SEQ ID NO: 9)
HW[FL]DX[WF],
(SEQ ID NO: 10)
ILQAD,
(SEQ ID NO: 11)
LHADXXF,
(SEQ ID NO: 12)
[IVTP]LHAD,
(SEQ ID NO: 13)
TLXAD[RSQ],
(SEQ ID NO: 14)
KXIPAE,
(SEQ ID NO: 15)
[ED]XAGYN,
(SEQ ID NO: 16)
[EDPA]X[AG][GA]YN,
(SEQ ID NO: 17)
C[RL]XTDC,
(SEQ ID NO: 18)
[LIV]L[QH]AE,
(SEQ ID NO: 19)
LPADR,
(SEQ ID NO: 20)
[ML]XXLSAD,
(SEQ ID NO: 21)
[FWY]XP[RV]AD,
(SEQ ID NO: 22)
AD[QR]E[HQ],
(SEQ ID NO: 23)
[FYL]XSAQP,
(SEQ ID NO: 24)
[SD]AQPX[WF],
(SEQ ID NO: 25)
[TS]AQPXX[WR],
(SEQ ID NO: 26)
MAGM[ED],
(SEQ ID NO: 27)
CMAGM,
(SEQ ID NO: 28)
CMAGE,
(SEQ ID NO: 29)
GPAG[EQ],
(SEQ ID NO: 30)
PAAG[TD],
(SEQ ID NO: 31)
PAG[TD]XXX[WRSHTG],
(SEQ ID NO: 32)
PA[GA][TD]XW,
(SEQ ID NO: 33)
[AP]AGT[MLI],
(SEQ ID NO: 34)
CXDPQC,
(SEQ ID NO: 35)
[LF]EDKS,
and
(SEQ ID NO: 36)
SXNLEQ.

Also provided for herein is a peptide library composition comprising: a) a library of nucleic acid sequences encoding a library of peptides, wherein the library of peptides comprises at least one distinct peptide, wherein the at least one distinct peptide comprises a binding motif, wherein the binding motif is selected from a motif group consisting of: [FM]TEX[FY]N (SEQ ID NO:4), KXGHDXC (SEQ ID NO:5), KGGXDX[IV] (SEQ ID NO:6), KXHG[IV]F (SEQ ID NO:7), HWFDXW (SEQ ID NO:8), HW[FL]DX[WF] (SEQ ID NO:9), ILQAD (SEQ ID NO:10), LHADXXF (SEQ ID NO:11), [IVTP]LHAD (SEQ ID NO:12), TLXAD[RSQ] (SEQ ID NO:13), KXIPAE (SEQ ID NO:14), [ED]XAGYN (SEQ ID NO:15), [EDPA]X[AG][GA]YN (SEQ ID NO:16), C[RL]XTDC (SEQ ID NO:17), [LIV]L[QH]AE (SEQ ID NO:18), LPADR (SEQ ID NO:19), [ML]XXLSAD (SEQ ID NO:20), [FWY]XP[RV]AD (SEQ ID NO:21), AD[QR]E[HQ] (SEQ ID NO:22), [FYL]XSAQP (SEQ ID NO:23), [SD]AQPX[WF] (SEQ ID NO:24), [TS]AQPXX[WR] (SEQ ID NO:25), MAGM[ED] (SEQ ID NO:26), CMAGM (SEQ ID NO:27), CMAGE (SEQ ID NO:28), GPAG[EQ] (SEQ ID NO:29), PAAG[TD] (SEQ ID NO:30), PAG[TD]XXX[WRSHTG] (SEQ ID NO:31), PA[GA][TD]XW (SEQ ID NO:32), [AP]AGT[MLI] (SEQ ID NO:33), CXDPQC (SEQ ID NO:34), [LF]EDKS (SEQ ID NO:35), and SXNLEQ (SEQ ID NO:36); and b) one or more display surfaces, wherein the at least one distinct peptide is capable of being expressed, and wherein each of the one or more display surfaces is configured to display one or more copies of at least one of the at least one distinct peptide.

In some aspects, the at least one distinct peptide comprising the binding motif LHADXXF (SEQ ID NO: 11) comprises the amino acid sequence EAKILHADLTEK (SEQ ID NO:37). In some aspects, the at least one distinct peptide comprising the binding motif HWFDXW (SEQ ID NO:8) comprises the amino acid sequence selected from the group consisting of: SNPHWFDTWIRV (SEQ ID NO:38), APRKAIPAENRL (SEQ ID NO:39), TKADIAGYNIVD (SEQ ID NO:40), NIKGGYDILTIA (SEQ ID NO:41), and combinations thereof. In some aspects, the at least one distinct peptide comprising the binding motif selected from the group consisting of: MAGM[ED] (SEQ ID NO:26), CMAGM (SEQ ID NO:27), CMAGE (SEQ ID NO:28), GPAG[EQ] (SEQ ID NO:29); PA[GA][TD]XW (SEQ ID NO:32); PAAG[TD] (SEQ ID NO:30): [AP]AGT[MLI] (SEQ ID NO:33) comprises the amino acid sequence HQGWAAGTIVVG (SEQ ID NO:42) and/or VWGLGPAGQWDT (SEQ ID NO:43).

In some aspects, the at least one distinct peptide comprising the binding motif KXHG[IV]F (SEQ ID NO:7) comprises the amino acid sequence selected from the group consisting of: KWPGIFSMPPMD (SEQ ID NO:44), RSQKQQGIFSFA (SEQ ID NO:45), KHFGIFSEDSWQ (SEQ ID NO:46), and combinations thereof. In some aspects, the at least one distinct peptide comprising the binding motif [FM]TEX[FY]N (SEQ ID NO:4) comprises the amino acid sequence selected from the group consisting of: HYTEYFNYDPRK (SEQ ID NO:47), GGRWQYTEDFN (SEQ ID NO:48), ALQDWHFTEQYN (SEQ ID NO:49), and combinations thereof.

In some aspects, the composition further comprises one or more display surfaces, wherein the at least one distinct peptide is capable of being expressed, and wherein each of the one or more display surfaces is configured to display one or more copies of at least one of the at least one distinct peptides.

In some aspects, the library of peptides comprises at least 2 distinct peptides each comprising a distinct binding motif, wherein each distinct binding motif is selected from the motif group. In some aspects, the library of peptides comprises at least 3, at least 4, or at least 5 distinct peptides each comprising a distinct binding motif, wherein each distinct binding motif is selected from the motif group. In some aspects, the library of peptides comprises at least 6, at least 7, at least 8, or at least 9 distinct peptides each comprising a distinct binding motif, wherein each distinct binding motif is selected from the motif group. In some aspects, the library of peptides comprises at least 10, at least 15, at least 20, at least 25, or at least 30 distinct peptides each comprising a distinct binding motif, wherein each distinct binding motif is selected from the motif group.

In some aspects, the at least one distinct peptide is capable of binding to an antibody associated with a Leptospirosis infection.

In some aspects, the one or more display surfaces comprises a single display surface. In some aspects, the single display surface comprises a solid support. In some aspects, each of the at least one distinct peptides is attached at a defined location on the single display surface. In some aspects, the single display surface is a peptide microarray. In some aspects, the defined location on the peptide microarray is a defined grid location on the peptide microarray. In some aspects, the single display surface is a multi-well plate. In some aspects, the defined location in the multi-well plate is a defined well in the multi-well plate. In some aspects, the single display surface comprises a lateral flow assay. In some aspects, the defined location in the lateral flow assay is a defined location on a capillary membrane.

In some aspects, the one or more display surfaces comprises a plurality of display surfaces. In some aspects, the plurality of display surfaces is a plurality of biological entity surfaces. In some aspects, the biological entity comprises a cell, a virus, or a bacteriophage. In some aspects, the at least one distinct peptide is encoded by one or more distinct nucleic acid sequences capable of expressing the at least one distinct peptide in the plurality of biological entities. In some aspects, the at least one distinct peptide comprises a surface display peptide sequence configured to be expressed as part of the peptide and capable of directing the peptides for display on the biological entity surface. In some aspects, the biological entity is an Escherichia coli cell. In some aspects, the at least one distinct peptide comprises at least a portion of an Escherichia coli eCPX scaffold. In some aspects, the plurality of display surfaces is a plurality of microparticle surfaces. In some aspects, each of the plurality of display surfaces comprises a single species of the at least one distinct peptide.

In some aspects, the library of peptides is a random peptide library.

In some aspects, the library of peptides is a non-random peptide library. In some aspects, the non-random library is biased to represent epitopes correlated with one or more diseases or conditions, and wherein at least one of the one or more diseases or conditions is a Leptospirosis infection. In some aspects, the biased representation is at least greater than a representation in a random peptide library. In some aspects, the greater representation is statistically significant, wherein the statistical significance has a p-value less than 0.05.

In some aspects, each of the peptides in the library of peptides is at least 5, 6, 7, 8, 9, 10, 11, 12, 13, 14, 15, 16, 17, 18, 19, or 20 amino acids in length.

In some aspects, each of the peptides in the library of peptides is an identical defined length. In some aspects, the identical defined length is 5, 6, 7, 8, 9, 10, 11, 12, 13, 14, 15, 16, 17, 18, 19, or 20 amino acids in length. In some aspects, the identical defined length is 12 amino acids in length.

In some aspects, two or more of the at least one distinct peptides or two or more of the library of peptides are concatenated. In some aspects, each of the at least one distinct peptides or each of the library of peptides are concatenated.

In some aspects, the library of peptides is selected from the group consisting of: a bacterial expression library, a yeast expression library, a bacteriophage expression library, a ribosomal expression library, and a mammalian expression library.

In some aspects, the one or more display surfaces or the distinct peptides is bound or capable of being bound directly or indirectly to a capture entity. In some aspects, the capture entity comprises a solid support selected from the group consisting of: an agarose bead, a sepharose bead, a magnetic bead, and a resin. In some aspects, the capture entity comprises a binding moiety that binds directly to the one or more display surfaces or the distinct peptides. In some aspects, the capture entity comprises a binding moiety that binds a binding molecule that binds to the one or more display surfaces or at least one of the distinct peptides. In some aspects, the binding molecule that binds to at least one of the distinct peptides is an antibody from a biological sample. In some aspects, the binding moiety is selected from the group consisting of: Protein A, Protein G, Protein A/G, and an antibody or antigen binding fragment thereof. In some aspects, the antibody or antigen binding fragment thereof comprises an anti-immunoglobulin antibody. In some aspects, the binding moiety comprises streptavidin and the one or more display surfaces, the distinct peptide, or the binding molecule that binds to the one or more display surfaces or at least one of the distinct peptides further comprises biotin. In some aspects, the capture entity further comprises a fluorophore.

In some aspects, displaying comprises presentation of each peptide for direct binding by a binding molecule. In some aspects, the binding molecule is selected from the group consisting of: an antibody or antigen-binding fragment thereof, an aptamer, a peptoid, and an affibody. In some aspects, the binding molecule is an antibody or antigen-binding fragment thereof.

In some aspects, each of the nucleic acid sequences encoding the library of peptides and each of the control binding targets is operably linked to at least one promoter. In some aspects, the at least one promoter is a constitutive promoter. In some aspects, the at least one promoter is an inducible promoter.

In some aspects, the nucleic acid sequences encoding the library of peptides further comprise polymerase chain reaction (PCR) primer target sequences configured for amplification of the nucleic acid sequences encoding the library of peptides.

Also provided herein is a library composition comprising two or more of the peptide expression library compositions described herein. In some aspects, each of the two or more of the peptide expression library compositions is in a separate container. In some aspects, the container is selected from the group consisting of: a well in a multi-well plate, a microcentrifuge tube, a test tube, a tube, and a PCR tube. In some aspects, the two or more of the peptide expression library compositions is 2, 3, 4, 5, 6, 7, 8, 9, 10-15, 16-24, 24-48, 48-96, or 96-384 peptide expression library compositions. In some aspects, the two or more of the peptide expression library compositions is at least 10, at least 20, at least 50, at least 100, at least 200, at least 300, at least 500, at least 1000, or at least 2000 expression library compositions.

Also provided herein is a kit comprising any of the compositions described herein and instructions for use.

Also provided herein is a method of manufacturing any of the compositions or kits described herein.

Also provided herein is a method of determining the presence of specimen antibodies specific for Leptospirosis in a biological sample comprising: contacting any one of the peptide expression library described herein with a biological sample, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope; measuring the binding between the at least one distinct peptide and the specimen antibodies; and determining specimen antibodies specific for Leptospirosis are present in the biological sample when binding between at least one of the at least one distinct peptide and the specimen antibodies is detected.

Also provided herein is a method of treating a subject known or suspected of suffering from a Leptospirosis infection comprising assessing or having assessed whether the subject suffers from a Leptospirosis infection, wherein the assessment comprises: contacting any one of the peptide expression library described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope; measuring the binding between the at least one distinct peptide and the specimen antibodies; and assessing the subject as: i) positive for the Leptospirosis infection, or ii) negative for the Leptospirosis infection; and administering or having administered a therapy to treat the Leptospirosis infection when the subject when the subject is assessed as positive.

Also provided herein is a method of determining whether a subject is a candidate for treatment of a Leptospirosis infection comprising assessing or having assessed whether the subject suffers from a Leptospirosis infection, wherein the assessment comprises: contacting any one of the peptide expression library described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope; measuring the binding between the at least one distinct peptide and the specimen antibodies; and diagnosing the subject as: i) positive for the Leptospirosis infection, or ii) negative for the Leptospirosis infection, determining the subject is a candidate for a treatment of the Leptospirosis infection when the subject is assessed as positive.

Also provided herein is a method of diagnosing a Leptospirosis infection in a subject comprising: contacting any one of the peptide expression library described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope; measuring the binding between the at least one distinct peptide and the specimen antibodies; and diagnosing the subject as: i) positive for the Leptospirosis infection, or ii) negative for the Leptospirosis.

In some aspects, the assessing or diagnosing comprises assessing or diagnosing the subject as: i) positive for the Leptospirosis infection when binding between at least one of the at least one distinct peptides and the specimen antibodies is detected, or ii) negative for the Leptospirosis infection when binding between less than at least one of the at least one distinct peptides and the specimen antibodies is detected.

In some aspects, the assessing or diagnosing comprises assessing or diagnosing the subject as: i) positive for the Leptospirosis infection when binding between at least 2, at least 3, at least 4, or at least 5 distinct peptides of the at least one distinct peptides and the specimen antibodies is detected, or ii) negative for the Leptospirosis infection when binding between less than at least 2, at least 3, at least 4, or at least 5 distinct peptides, respectively, of the at least one distinct peptides and the specimen antibodies is detected.

In some aspects, the assessing or diagnosing comprises assessing or diagnosing the subject as: i) positive for the Leptospirosis infection when binding between at least 6, at least 7, at least 8, at least 9, or at least 10 distinct peptides of the at least one distinct peptides and the specimen antibodies is detected, or ii) negative for the Leptospirosis infection when binding between less than at least 6, at least 7, at least 8, at least 9, or at least 10 distinct peptides, respectively, of the at least one distinct peptides and the specimen antibodies is detected.

In some aspects, the measuring comprises calculating a z-score for binding between the at least one distinct peptide and the specimen antibodies and the assessing or diagnosing comprises assessing or diagnosing the subject as: i) positive for the Leptospirosis infection when the z-score meets or exceeds a threshold value, or ii) negative for the Leptospirosis infection when the z-score is less than the threshold value.

In some aspects, the method further comprises determining amino acid sequences of peptides in the library composition bound by the specimen antibodies. In some aspects, determining the amino acid sequences comprises isolating the specimen binding molecules bound to the one or more display surfaces and sequencing the nucleic acids encoding the distinct peptide to determine the amino acid sequences bound by the specimen binding molecules. In some aspects, the method further comprises determining binding motifs of peptides in the library composition bound by the specimen antibodies using a clustering algorithm. In some aspects, determining the amino acid sequences comprises isolating the specimen binding molecules bound to the one or more display surfaces and sequencing the nucleic acids encoding the distinct peptide to determine the amino acid sequences bound by the specimen binding molecules: i) determining a binding location of the specimen binding molecule on the one or more display surfaces; ii) correlating the binding location with the defined location of the distinct peptides to determine the amino acid sequences bound by the specimen binding molecules.

In some aspects, the sample is selected from the group consisting of: serum, blood, saliva, urine, tissue, tissue homogenates, stool, spinal fluid, and lysate derived from animal sources. In some aspects, the sample is human serum. In some aspects, the human serum is from a subject suspected of having a Leptospirosis infection. In some aspects, the subject is suspected of having an acute Leptospirosis infection. In some aspects, the subject is suspected of having a convalescent Leptospirosis infection.

In some aspects, the binding molecules in the sample are antibodies. In some aspects, the antibodies comprise one or more antibodies known or suspected to recognize antigens or epitopes correlating with a Leptospirosis infection.

In some aspects, the contacting comprises mixing the sample, the peptide expression library composition, and the control binding molecule in a container selected from the group consisting of: a well in a multi-well plate, a microcentrifuge tube, a test tube, a tube, and a PCR tube.

In some aspects, the isolating step is selected from the group consisting of: magnetic isolation, bead centrifugation, resin centrifugation, and flow cytometry. In some aspects, the isolating step comprises contacting the sample with a capture entity. In some aspects, the capture entity is selected from the group consisting of: an agarose bead, a sepharose bead, a magnetic bead, and a resin. In some aspects, the capture entity comprises a binding moiety. In some aspects, the binding moiety is selected from the group consisting of: Protein A, Protein G, Protein A/G, and an antibody or antigen binding fragment thereof. In some aspects, the antibody or antigen binding fragment thereof comprises an anti-immunoglobulin antibody. In some aspects, the binding moiety comprises streptavidin and the specimen binding molecules further comprises biotin.

In some aspects, when the one or more display surfaces is a biological entity surface, the determining step further comprises the steps of: 1) purifying or having purified polynucleotides from the biological entity, wherein the polynucleotides comprise the nucleic acid sequences encoding the peptide bound by the isolated specimen binding molecules; 2) amplifying or having amplified the nucleic acid sequences encoding the peptide bound by the isolated specimen binding molecules; and 3) sequencing or having sequenced the amplified polynucleotides. In some aspects, the amplifying step further comprises adding a sample identifying nucleic acid sequence to a terminus of the amplified polynucleotides, wherein the sample identifying nucleic acid sequence is unique to the sample. In some aspects, the method further comprises the step of determining the identity of the sample identifying nucleic acid sequences.

In some aspects, the method has a sensitivity of at least 60%, at least 70%, at least 80%, at least 90%, or at least 95%. In some aspects, the method has a sensitivity of at least 80%. In some aspects, the method has a sensitivity of at least 90%. In some aspects, the method has a sensitivity of at least 94%. In some aspects, the method has a sensitivity of at least 97%. In some aspects, the method has a sensitivity of 100% In some aspects, the method has a sensitivity of at least 91%, at least 92%, at least 93%, at least 94%, at least 95%, at least 96%, at least 97%, at least 98%, at least 99%, or 100%. In some aspects, the biological sample is from a subject suspected of having an acute Leptospirosis infection, and wherein the method has a sensitivity of at least 60%, optionally wherein the binding motifs are selected from IgG isotype specific binding motifs. In some aspects, the biological sample is from a subject suspected of having an acute Leptospirosis infection, and wherein the method has a sensitivity of at least 80%, optionally wherein the binding motifs are selected from both IgG and IgM isotype specific binding motifs. In some aspects, the biological sample is from a subject suspected of having an acute Leptospirosis infection, and wherein the method has a sensitivity of at least 90%, optionally wherein the binding motifs are selected from IgM isotype specific binding motifs. In some aspects, the biological sample is from a subject suspected of having an acute Leptospirosis infection, and wherein the method has a sensitivity of at least 90%, optionally wherein the binding motifs are selected from both IgG and IgM isotype specific binding motifs. In some aspects, the method has a sensitivity greater than a microscopic agglutination test. In some aspects, the method has a sensitivity of at least 60%, at least 61%, at least 62%, at least 63%, at least 64%, at least 65%, at least 66%, at least 67%, at least 68%, at least 69%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, or at least 89%.

In some aspects, the method has a specificity greater than a microscopic agglutination test. In some aspects, the method has a specificity of at least 90%, at least 91%, at least 92%, at least 93%, at least 94%, at least 95%, at least 96%, at least 97%, at least 98%, or at least 99%. In some aspects, the method has a specificity of at least 96%. In some aspects, the method has a specificity of at least 97%. In some aspects, the method has a specificity of at least 99%. In some aspects, the binding motifs are selected from both IgG and IgM isotype specific binding motifs, and wherein the method has a specificity of at least 96%. In some aspects, the binding motifs are selected from IgM isotype specific binding motifs, and wherein the method has a specificity of at least 97%. In some aspects, the binding motifs are selected from IgG isotype specific binding motifs, and wherein the method has a specificity of at least 99%. In some aspects, the binding motifs are selected from IgG isotype specific binding motifs, and wherein the method has a specificity of at least 99.5%.

In some aspects, the method has a sensitivity of at least 60% and a specificity of at least 90%. In some aspects, the method has a sensitivity of at least 60% and a specificity of at least 95%. In some aspects, the method has a sensitivity of at least 80% and a specificity of at least 90%. In some aspects, the method has a sensitivity of at least 80% and a specificity of at least 95%. In some aspects, the method has a sensitivity of at least 90% and a specificity of at least 90%. In some aspects, the method has a sensitivity of at least 90% and a specificity of at least 95%.

In some aspects, calculating the z-score comprises calculating a composite z-score for binding between each of the at least one distinct peptides specific for Leptospirosis and the specimen antibodies.

### 5. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A presents enrichment for the IgG motif HWFDXW (SEQ ID NO:8) in Leptospirosis (n=60) relative to controls (n=305).

FIG. 1B presents enrichment for the IgG motif HW[FL]DX[WF] (SEQ ID NO:9) in Leptospirosis (n=60) relative to controls (n=305).

FIG. 1C presents enrichment for the IgG motif ILQAD (SEQ ID NO:10) in Leptospirosis (n=60) relative to controls (n=305).

FIG. 1D presents enrichment for the IgG motif LHADXXF (SEQ ID NO:11) in Leptospirosis (n=60) relative to controls (n=305).

FIG. 1E presents enrichment for the IgG motif [IVTP]LHAD (SEQ ID NO:12) in Leptospirosis (n=60) relative to controls (n=305).

FIG. 1F presents enrichment for the IgG motif TLXAD[RSQ] (SEQ ID NO: 13) in Leptospirosis (n=60) relative to controls (n=305).

FIG. 1G presents enrichment for the IgG motif [FM]TEX[FY]N (SEQ ID NO:4) in Leptospirosis (n=60) relative to controls (n=305).

FIG. 1H presents enrichment for the IgG motif KXGHDXC (SEQ ID NO:5) in Leptospirosis (n=60) relative to controls (n=305).

FIG. 1I presents enrichment for the IgG motif KGGXDX[IV] (SEQ ID NO:6) in Leptospirosis (n=60) relative to controls (n=305).

FIG. 1J presents enrichment for the IgG motif KXHG[IV]F (SEQ ID NO:7) in Leptospirosis (n=60) relative to controls (n=305).

FIG. 1K presents enrichment for the IgG motif KXIPAE (SEQ ID NO:14) in Leptospirosis (n=60) relative to controls (n=305).

FIG. 1L presents enrichment for the IgG motif [ED]XAGYN (SEQ ID NO:15) in Leptospirosis (n=60) relative to controls (n=305).

FIG. 1M presents enrichment for the IgG motif [EDPA]X[AG][GA]YN (SEQ ID NO:16) in Leptospirosis (n=60) relative to controls (n=305).

FIG. 2A presents enrichment for the IgM motif C[RL]XTDC (SEQ ID NO:17) in Leptospirosis (n=60) relative to controls (n=317).

FIG. 2B presents enrichment for the IgM motif [LIV]L[QH]AE (SEQ ID NO:18) in Leptospirosis (n=60) relative to controls (n=317).

FIG. 2C presents enrichment for the IgM motif LPADR (SEQ ID NO:19) in Leptospirosis (n=60) relative to controls (n=317).

FIG. 2D presents enrichment for the IgM motif [ML]XXLSAD (SEQ ID NO:20) in Leptospirosis (n=60) relative to controls (n=317).

FIG. 2E presents enrichment for the IgM motif [FWY]XP[RV]AD (SEQ ID NO:21) in Leptospirosis (n=60) relative to controls (n=317).

FIG. 2F presents enrichment for the IgM motif AD[QR]E[HQ] (SEQ ID NO:22) in Leptospirosis (n=60) relative to controls (n=317).

FIG. 2G presents enrichment for the IgM motif [FYL]XSAQP (SEQ ID NO:23) in Leptospirosis (n=60) relative to controls (n=317).

FIG. 2H presents enrichment for the IgM motif [SD]AQPX[WF] (SEQ ID NO:24) in Leptospirosis (n=60) relative to controls (n=317).

FIG. 2I presents enrichment for the IgM motif [TS]AQPXX[WR] (SEQ ID NO:25) in Leptospirosis (n=60) relative to controls (n=317).

FIG. 2J presents enrichment for the IgM motif MAGM[ED] (SEQ ID NO:26) in Leptospirosis (n=60) relative to controls (n=317).

FIG. 2K presents enrichment for the IgM motif CMAGM (SEQ ID NO:27) in Leptospirosis (n=60) relative to controls (n=317).

FIG. 2L presents enrichment for the IgM motif CMAGE (SEQ ID NO:28) in Leptospirosis (n=60) relative to controls (n=317).

FIG. 2M presents enrichment for the IgM motif GPAG[EQ] (SEQ ID NO:29) in Leptospirosis (n=60) relative to controls (n=317).

FIG. 2N presents enrichment for the IgM motif PAAG[TD] (SEQ ID NO:30) in Leptospirosis (n=60) relative to controls (n=317).

FIG. 2O presents enrichment for the IgM motif PAG[TD]XXX[WRSHTG] (SEQ ID NO:31) in Leptospirosis (n=60) relative to controls (n=317).

FIG. 2P presents enrichment for the IgM motif PA[GA][TD]XW (SEQ ID NO:32) in Leptospirosis (n=60) relative to controls (n=317).

FIG. 2Q presents enrichment for the IgM motif [AP]AGT[MLI] (SEQ ID NO:33) in Leptospirosis (n=60) relative to controls (n=317).

FIG. 2R presents enrichment for the IgM motif CXDPQC (SEQ ID NO:34) in Leptospirosis (n=60) relative to controls (n=317).

FIG. 2S presents enrichment for the IgM motif [LF]EDKS (SEQ ID NO:35) in Leptospirosis (n=60) relative to controls (n=317).

FIG. 2T presents enrichment for the IgM motif SXNLEQ (SEQ ID NO:36) in Leptospirosis (n=60) relative to controls (n=317).

FIG. 3A presents IgG epitope motif panel composite z-scores for acute and convalescent patient samples. The cutoff for positivity was set at 20 for IgG. MAT and IgM SERA phenotype are also indicated for each sample. Also shown is sensitivity by MAT and SERA panels for each of the blood draw panels, as well as IgG SERA specificity.

FIG. 3B presents IgM epitope motif panel composite z-scores for acute and convalescent patient samples for both discovery panels and validations panels. The cutoff for positivity was set at 25 for IgM. MAT and IgG SERA phenotype are also indicated for each sample. Also shown is sensitivity by MAT and SERA panels for each of the blood draw panels, as well as IgM SERA specificity.

FIG. 4 presents iterative performance sensitivity analysis when combining IgG (right panel) and IgM (left panel) Leptospirosis-specific epitope motifs.

### 6. DETAILED DESCRIPTION

### 6.1. Definitions

Terms used in the claims and specification are defined as set forth below unless otherwise specified.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Numerical values provided herein can sometimes be considered to be modified by the term about, where context makes clear that the ranges encompassed by the modification are consistent with operability of the invention and definiteness of the claims.

The term “motif” as used herein comprises an amino acid sequence pattern, which comprises preferred amino acids at each position of a peptide sequence. For example, HW[FL]DX[WF] (SEQ ID NO:9) where “X” is any amino acid and each letter corresponds to the conventional one-letter amino acid code. The notation [XYZ] within a motif means that the indicated position comprises one amino acid that is selected from “X or Y or Z”. Motifs may alternatively be presented graphically as a sequence “logo,” wherein the frequencies of occurrence of individual amino acids at each position in a motif are represented by the height of the character (e.g. one letter amino acid code) at that position. A larger letter indicates a higher frequency of occurrence.

The term “epitope” refers to the part of an antigen molecule/s to which a binding molecule (e.g., an antibody) attaches itself. For example, in the case of a protein antigen, the epitope can be the amino acid sequence or protein structural region to which the binding molecule (e.g., antibody) binds, in other words the amino acid sequence or protein structural region responsible for mediating a non-covalent interaction between a binding molecule and its cognate target.

The term “epitope repertoire” as used herein comprises the set of all antigens recognized, or bound by, by a binding molecules (e.g., antibodies) within a sample, or group of samples. For example, the epitope repertoire may refer to the set of all peptides or antigens recognized, or bound by, by binding molecules (e.g., antibodies) within a sample, or group of samples.

The term “enrichment” or “enriched” as used herein refers to the number of observations of a peptide, pattern, or motif within an epitope repertoire divided by the number expected within a random dataset of equivalent size. For example, in a hypothetical 9-mer peptide library (-XXXXXXXXX-), where X is any amino acid, the pattern QPXXPFX[ED] (SEQ ID NO:3) is expected to occur once in every 800,000 ((1aa/20aa)4×(2aa/20aa)×2) random sequences (aa=amino acid). If 4 million sequences were determined, then one would expect to observe five (5) occurrences (i.e., once in every 800,000 sequences). As an example, if the pattern was actually observed in 50 unique peptides sequences (i.e. 50 observations) in an epitope repertoire, then the pattern would be “enriched” by 10-fold versus random.

The term “threshold” as used herein refers to the magnitude or intensity that must be exceeded for a certain reaction, phenomenon, result, or condition to occur or be considered relevant. For example, the threshold can be a numerical value above which enrichment is considered relevant. The relevance can depend on context, e.g., it may refer to a positive, reactive or statistically significant relevance.

As used here, the terms “massively parallel signature sequencing” (MPSS) or “next generation sequencing” (NGS) and the like are used interchangeably to refer to high throughput nucleic acid sequencing (HTS) approaches. Platforms for NGS that rely on different sequencing technologies are commercially available from a number of vendors such as Pacific Biosciences, Ion Torrent from Thermo Fisher, 454 Life Sciences, Illumina, Inc. (e.g., MiSeq, NextSeq, HiSeq) and Oxford Nanopore. For a review of NGS technologies, see, e.g., van Dijk E L et al. Ten years of next-generation sequencing technology. Trends Genet. 2014 September; 30(9):418-26, herein incorporated by reference in its entirety for all purposes.

The term “clustering algorithm” as used herein refers to a computational algorithm used to perform “cluster analysis.” Cluster analysis or clustering is the task of grouping a set of objects in such a way that objects in the same group (called a cluster) are more similar (in some sense or another) to each other than to those in other groups (clusters). A variety of clustering algorithms are known to those of skill in the art. See, e.g., Bailey et al. (Fitting a mixture model by expectation maximization to discover motifs in biopolymers. Proc Int Conf Intell Syst Mol Biol, 1994. 2: p. 28-36); Amstutz et al. (In vitro display technologies: novel developments and applications. Curr Opin Biotechnol, 2001. 12(4): p. 400-5); and Gould-Rothberg et al. (Massively parallel (“next-generation”) DNA sequencing. Clin Chem, 2015. 61(7): p. 997-8.), each herein incorporated by reference in their entirety for all purposes.

The term “disease” refers to an abnormal condition affecting the body of an organism. The term “disorder” refers to a functional abnormality or disturbance. The terms disease or disorder are used interchangeably herein unless otherwise noted or clear given the context in which the term is used. The terms disease and disorder may also be referred to collectively as a “condition.”

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al.).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect.

The term “therapeutically effective amount” is an amount that is effective to ameliorate a symptom of a disease. In some contexts, a therapeutically effective amount can be a “prophylactically effective amount” as prophylaxis can be considered therapy, provided such interpretation does not adversely impact any determination of the validity of any claim for any reason.

It must be noted that, as used in the specification and the appended claims, the singular forms “a,” “an,” and “the” include plural referents unless the context clearly dictates otherwise.

### 6.2. Peptide Display Systems

In one aspect, peptide display systems are provided. In a particular aspect, peptide display systems comprising peptides corresponding to epitopes associated with Leptospirosis are provided. A peptide display system has at a minimum the peptides to be displayed, e.g., peptides corresponding to epitopes associated with Leptospirosis, and one or more display surfaces.

### 6.2.1. Peptides

A peptide display system can include one or more distinct peptides. The terms “peptide,” “polypeptide,” “amino acid sequence,” “peptide sequence,” and “protein” are used interchangeably to refer to two or more amino acids linked together and imply no particular length. As used herein, a “distinct peptide” refers to a peptide having a specific amino acid sequence composition. Amino acids can be naturally occurring or synthetic (e.g., non-natural amino acids or amino acid analogs). Peptides can be naturally occurring peptides, e.g., a peptide having an amino acid sequence known to occur entirely in an organism and without additional non-natural modifications. Peptides can be non-naturally occurring peptides, e.g., a peptide having (1) an amino acid sequence not known to occur in its entirety in an organism and/or (2) containing non-natural modifications, such as non-natural amino acids or amino acid analogs. Amino acids and peptides can also comprise, or be further modified to comprise, reactive groups, such as reactive groups for attaching amino acids or peptides to display surfaces (e.g., solid substrates), reactive groups for labeling amino acids or peptides, or reactive groups for attaching other moieties of interest to amino acids or peptides. Reactive groups include, but are not limited to, chemically-reactive groups such as reactive thiols (e.g., maleimide based reactive groups), reactive amines (e.g., N-hydroxysuccinimide based reactive groups), “click chemistry” groups (e.g., reactive alkyne groups), and aldehydes bearing formylglycine (FGly).

A peptide display system includes at least one distinct peptide. A peptide display system can include one or more distinct peptides. A peptide display system can have any number of distinct peptides useful for the present invention, such as any number of distinct peptides suitable for displaying on a display surface such that the distinct peptides are capable of recognition by a binding molecule. It is understood that references to numbers of distinct peptides does not refer to the absolute quantity (i.e., copies, occurrences, or concentration) of each distinct peptide, but instead to the number of distinct peptide species. In some examples, the number of distinct peptides possible is determined by the physical limitations of a display surface, such as the size of a peptide microarray or number of wells in a multi-well plate.

A peptide display system can include two or more distinct peptides. A peptide display system can include at least 3, at least 4, or at least 5 distinct peptides. A peptide display system can include at least 6, at least 7, at least 8, at least 9, or at least 10 distinct peptides. A peptide display system can include at least 3 distinct peptides. A peptide display system can include at least 4 distinct peptides. A peptide display system can include at least 5 distinct peptides. A peptide display system can include at least 6 distinct peptides. A peptide display system can include at least 7 distinct peptides. A peptide display system can include at least 8 distinct peptides. A peptide display system can include at least 9 distinct peptides. A peptide display system can include at least 10 distinct peptides. A peptide display system can include at least 15, at least 20, at least 25, or at least 30 distinct peptides. A peptide display system can include at least 40, at least 50, at least 60, at least 70, at least 80, at least 90, or at least 100 distinct peptides. A peptide display system can include at least 200, at least 300, at least 400, at least 500, at least 600, at least 700, at least 800, at least 900, or at least 1000 distinct peptides. A peptide display system can include at least 300,000 distinct peptides. A peptide display system can include 300,000 or fewer distinct peptides. A peptide display system can include 384 or fewer, 96 or fewer, 48 or fewer, 24 or fewer, or 12 or fewer distinct peptides.

Typically, peptides in a peptide display system and/or a library can also all fall within a range of lengths. For example, the peptides, or epitope portions thereof, in a peptide display system and/or a library may be different lengths, but all fall within a defined range of lengths. The selected range can be any length useful for the present invention, such as any length suitable for displaying an epitope sequence on a display surface such that it is capable of recognition by a binding molecule. The peptides in a peptide display system and/or a library can be at least 5, 6, 7, 8, 9, 10, 11, 12, 13, 14, 15, 16, 17, 18, 19, or 20 amino acids in length. The peptides in a peptide display system and/or a library can also be 5-30, 5-25, 5-20, 5-15, 5-10, 10-30, 10-25, 10-20, or 10-15 amino acids in length. The peptides in a peptide display system and/or a library can also be 7-14, 8-14, 9-14, 10-14, 11-14, 12-14, 7-13, 8-13, 9-13, 10-13, 11-13, 12-13, 7-12, 8-12, 9-12, 10-12, 11-12, 7-11, 8-11, 9-11, or amino acids in length. If desired, the peptides in a peptide display system and/or a library can also be greater than 30, greater than 40, greater than 50, greater than 75, greater than 100, greater than 200, or greater than 300 amino acids in length. The epitope portions of peptides in a peptide display system and/or a library can be at least 5, 6, 7, 8, 9, 10, 11, 12, 13, 14, 15, 16, 17, 18, 19, or 20 amino acids in length. The epitope portions of peptides in a peptide display system and/or a library can also be 5-30, 5-25, 5-20, 5-15, 5-10, 10-30, 10-25, 10-20, or 10-15 amino acids in length. The epitope portions of peptides in a peptide display system and/or a library can also be 7-14, 8-14, 9-14, 10-14, 11-14, 12-14, 7-13, 8-13, 9-13, 10-13, 11-13, 12-13, 7-12, 8-12, 9-12, 10-12, 11-12, 7-11, 8-11, 9-11, or amino acids in length. If desired, the epitope portions of peptides in a peptide display system and/or a library can also be greater than 30, greater than 40, greater than 50, greater than 75, greater than 100, greater than 200, or greater than 300 amino acids in length.

Peptides, or the epitope portions thereof, in a peptide display system and/or a library can also be an identical defined length, i.e., all the peptides in the a peptide display system and/or the library have the same number of amino acids. The defined length can be any length useful for the present invention, such as any length suitable for displaying an epitope sequence on a display surface such that it is capable of recognition by a binding molecule. The defined length can be 5, 6, 7, 8, 9, 10, 11, 12, 13, 14, 15, 16, 17, 18, 19, or 20 amino acids in length. In an illustrative non-limiting example, each distinct peptide in the peptide display system and/or the library is 12 amino acids in length. In another illustrative non-limiting example, each distinct peptide in the peptide display system and/or the library has a peptide portion that is 12 amino acids in length that is capable of recognition by a binding molecule (e.g., each distinct peptide has an epitope sequence that is 12 amino acids in length). The defined length (e.g., the defined length of 5, 6, 7, 8, 9, 10, 11, 12, 13, 14, 15, 16, 17, 18, 19, or 20 amino acids) can refer to only the epitope portion of the peptide, such as the epitope portion of a peptide in an E. coli surface display peptide library, where the full-length peptide also contains a cell surface display peptide (see Section 6.2.3). The defined length (e.g., the defined length of 5, 6, 7, 8, 9, 10, 11, 12, 13, 14, 15, 16, 17, 18, 19, or 20 amino acids) can refer to the length of the full-length peptide. The defined length (e.g., the defined length of 5, 6, 7, 8, 9, 10, 11, 12, 13, 14, 15, 16, 17, 18, 19, or 20 amino acids) can refer to the length of the full-length peptide, where the full-length peptide is the epitope portion of the peptide, such as in some instances the distinct peptides used in a peptide microarray.

Peptides, or the epitope portions thereof, in a peptide display system and/or a library can also be concatenated. For example, a concatenated peptide can have two or more distinct peptides regions capable of recognition by a binding molecule (e.g., two or more distinct epitope sequences or epitope motifs). The two or more distinct peptide regions of a concatenated peptide can be linearly linked in a N-terminal to C-terminal orientation. The two or more distinct peptide regions of a concatenated peptide can be directly linked to each other (i.e., a first distinct peptide sequence is immediately followed by a second distinct peptide sequence). The two or more distinct peptide regions of a concatenated peptide can be linked to each other by a peptide-linker sequence (i.e., a first distinct peptide region and a second distinct peptide region are separated by an intervening amino acid “linker” sequence not considered to be part of either distinct peptide sequence). A peptide display system and/or a library can include multiple distinct concatenated peptides, such as each concatenated peptide having a distinct and/or non-overlapping set of distinct peptide regions (e.g., distinct peptide regions corresponding to distinct epitope sequences or epitope motifs).

A peptide can include the specific amino acids or amino acid classes (e.g., hydrophobic, hydrophilic, acidic, basic, or steric amino acid) as described by a peptide motif sequence. A peptide can include the amino acids or amino acid classes as described by a peptide motif sequence where the full peptide sequence is not known to occur in its entirety in an organism. A peptide can include the amino acids or amino acid classes as described by a peptide motif sequence where the motif sequence is derived from an organism, while the full peptide sequence is not known to occur in its entirety in an organism. A peptide can include the amino acids or amino acid classes as described by a peptide motif sequence where the full peptide sequence is derived from an organism. A peptide can include the specific amino acids or amino acid classes representative of a motif class. As used herein, a “motif class” refers to peptide motif sequences that can include a common specific amino acid or amino acid class in at least 3 specific positions. As an illustrative non-limiting example, peptide motif sequences GPAG[EQ] (SEQ ID NO:29), PA[GA][TD]XW (SEQ ID NO:32), and [AP]AGT[MLI] (SEQ ID NO:33) can be considered as part of the same motif class given each share the option of the same amino acid sequence PAG. In general, a motif class represents peptide motif sequences that are known or are likely to encompass the same epitope derived from an organism.

### 6.2.2. Peptide Libraries

A peptide display system can include a library of peptides. As used herein, a “library of peptides” or a “peptide library” refers to a collection of a peptide fragments. Peptide libraries can be used for screening purposes. In general, a peptide library contains a large variety of distinct peptides. For example, the diversity of the library (i.e., the number of distinct peptides in a library and sometimes referred to as “complexity” of the library) can be more than 10⁴, more than 10⁵, more than 10⁶, more than 10⁷, more than 10⁸, more than 10⁹, more than 10¹⁰, or more than 10¹¹ distinct peptides. The library can be a random peptide library where the amino acid sequences are unbiased. A particular embodiment of a random/unbiased library is one constructed to represent all possible amino acid sequences of designated length(s). Peptide libraries can be produced by a peptide expression library (see Section 6.3) Peptide libraries include, but are not limited to, peptide libraries produced using bacterial expression libraries, yeast expression libraries, bacteriophage expression libraries, ribosome expression libraries, and mammalian expression libraries. Particular peptide libraries and peptide expression libraries useful for the present invention are described in more detail in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference in their entirety for all purposes.

A peptide library can also be a non-random library where the amino acid sequences are biased in their representation. A peptide library can also be a non-random library where the amino acid sequence motifs are biased in their representation. For example, a library can be biased to represent, over represent, predominantly represent, or only represent amino acid sequences, or amino acid sequence motifs, characteristic of a particular feature, such as epitopes or antigens associated with a particular disease (e.g., a bacterial infection, a viral infection, a parasitic infection, an autoimmune disorder, cancer, allergies etc.), condition, species (e.g., mammal, human, bacteria, virus etc.), protein, class of proteins, protein motif (e.g., phosphorylation motifs, binding motifs, protein domains, etc.), amino acid property (e.g., hydrophobic, hydrophilic, acidic, basic, or steric amino acid properties), or any other subset of amino acid sequences that is rationally designed. In an illustrative non-limiting example, a library is biased to represent epitopes associated with Leptospirosis. In another illustrative non-limiting example, a library is biased to represent epitopes associated with a panel of diseases, such as a panel including Leptospirosis. A library can be biased to also avoid certain amino acid sequences or motifs. A biased library can be said to be enriched for one or more particular sequences, peptides, patterns, or motifs. Bias can be quantitated using statistical measurements, such as determining the representation of a distinct peptide or motif relative to its expected representation by chance in a random library. The bias of peptide representation in a library can be statistically significant, such as a distinct peptide or peptide motif having a greater representation than in a random library of the same size with a statistical significance having a p-value less than 0.05.

Each distinct peptide in the library of peptides can share a common feature, such as all representing epitopes associated with Leptospirosis. A library can also have one or more groups of peptides in the library of peptides, each group independently sharing a common feature, such as each group independently representing epitopes associated with particular disease (e.g., a peptide panel representing a variety of diseases).

A peptide library can also combine the features of a non-random and random peptide library. For example, one or more select positions within an amino acid sequence may be a constant amino acid and other positions within the sequence may be fully random or biased based on other properties. In other examples, one or more select positions within an amino acid sequence may be selected from a defined subset of amino acids. One skilled in the art will appreciate that the various biases described can combined to achieve a desired purpose of the peptide library, such as a targeted screen.

### 6.2.3. Display Surfaces

A peptide display system has one or more display surfaces. As used herein, a “display surface” refers to any surface that can be configured to display (i.e., present) peptides in a manner suitable for recognition by their respective binding molecules, e.g., antibodies, such as antibodies present in a biological sample.

Display surfaces can be a biological entity surface (e.g., the outer membrane surface of a cell). Biological entities that can be used include, but are not limited to, a mammalian cell, a yeast, a bacteria, a virus, and a bacteriophage. The members of a library of peptides (e.g., candidate peptides) and/or selected peptide sequences (e.g., epitope motifs, such as epitopes associated with Leptospirosis) can be engineered to be expressed on the surface of a cell, such as constructing the library of nucleic acid sequences encoding the library of peptides or the nucleic acid sequences encoding the selected peptide sequences to also encode a cell surface display peptide sequence configured to be expressed as part of the peptide and capable of directing the peptides for display on the biological entity surface. Illustrative non-limiting examples of E. coli cell surface display systems and display surfaces are described in greater detail in in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference in their entirety for all purposes.

Display surfaces can include solid supports. As described herein, a “solid support” refers to a material to which other entities can be attached (i.e., immobilized) and typically refers to materials that are non-biological in nature (e.g., not the surface of an organism, such as a cell membrane, viral surface, or bacteriophage surface). Materials that can be used to produce solid supports include, but are not limited to, glass, functionalized glass, silicon, germanium, gallium arsenide, gallium phosphide, silicon dioxide, sodium oxide, silicon nitrade, nitrocellulose, nylon, polytetrafluoroethylene, polyvinylidendiflouride, polystyrene, polycarbonate, methacrylates, or combinations thereof. The solid support and/or the surface of the solid support can be homogeneous or heterogeneous, e.g., composed of a homogeneous or heterogeneous material.

In some embodiments, the display system uses single display surface. In some embodiments, the single surface is a solid support. Solid supports with a single surface can include, but are not limited to, a planar surface, e.g., a multi-well plate, peptide microarray (also sometimes referred to as a peptide chip) or lateral flow assay (also sometimes referred to as a lateral flow immunochromatographic assay or lateral low test). A planar surface can be flat, essentially flat, concave, or convex. Peptides can be attached to the single surface, e.g., a multi-well plate, peptide microarray, or lateral flow assay capillary membrane, such that each distinct peptide species is attached to the surface at a defined location (e.g., in a particular well, a particular grid location of a microarray, or a particular region of a capillary membrane) such that the defined location can be used as a reference for identification of the peptide species. Attachment of multiple distinct peptide species at defined locations can be said to be “arrayed” on the display surface or to form a “peptide array.”

In some embodiments, the display system uses a plurality of display surfaces. In some embodiments, the plurality of display surfaces are a plurality of solid supports. A plurality of solid supports can include, but are not limited to, a plurality of beads (also sometimes referred to as microbeads, microparticles, nanoparticles, or nanobeads). Examples of beads include, but are not limited to, agarose beads, polystyrene beads, magnetic beads (e.g., Dynabeads™ ThermoFisher or Pierce), polymers (e.g., dextran), synthetic polymers (e.g., Sepharose™), or any other bead suitable for displaying peptides. In some embodiments, the plurality of display surfaces are biological entity surfaces, such as a plurality of cells, viruses, bacteriophage, or ribosomes.

In some embodiments, each display surface (or defined location of an array surface) is configured to display one or more copies of a single species of a peptide (i.e., only distinct peptides having the same amino acid sequence). In a non-limiting illustrative example, a display surface is a multi-well plate well that contains multiple copies of a single species of a peptide, or display surface is a defined location of a peptide array grid that contains multiple copies of a single species of a peptide. In another non-limiting illustrative example, a composition has a plurality of beads or biological entities where each bead or biological entity displays multiples copies a single species of a peptide. In a further example, a display library can have a plurality of display surfaces, where each display surface is configured to display one or more copies of a single species of a peptide (i.e., a distinct peptide), and where multiple peptide species are represented by individual members of the plurality having a single distinct peptide species.

In some embodiments, each display surface is configured to display one or more copies of multiple distinct peptide species. In a non-limiting illustrative example, a composition has a single peptide microarray display surface where the peptide microarray displays multiples copies of multiple peptide species on the same surface.

In some embodiments, peptides are attached (also sometimes referred to as “immobilized”) to the display surfaces. Peptides can be attached either their N or C terminus. In some embodiments, proteins/peptides, nucleic acids, or both attached to the display surface and can be modified for use in the present invention. Concatenated peptides can be attached to a display surface. The display surfaces (e.g., the solid supports) and/or the peptides can be modified or functionalized to allow attachment of the peptide to the display surface. Methods of attaching proteins/peptides and nucleic acids are known to those skilled in the art and include, but are not limited to, use of chemically reactive groups such as reactive thiols (e.g., maleimide based reactive groups), reactive amines (e.g., N-hydroxysuccinimide based reactive groups), “click chemistry” groups (e.g., reactive alkyne groups), aldehydes bearing formylglycine (FGly) and other cognate modifications (e.g., biotin-streptavidin pairs, disulfide linkages, polyhistidine-nickel). Examples of modifications include chemical modifications that allow formation of covalent bonds with proteins (e.g., activated aldehyde groups) and modifications that specifically pair a modified display surface with a peptide having a cognate modification (e.g., biotin-streptavidin pairs, disulfide linkages, polyhistidine-nickel, or “click-chemistry” modifications such as azido-alkynyl pairs). Modifications can also include functional groups, such as an imide functional group, an amine functional group, a hydroxyl functional group, a carboxyl functional group, an aldehyde functional group, and/or a sulfhydryl functional group. Methods of attaching peptides to microarrays are known to those skilled in the art, for example, as described in more detail in Macbeath, G.; Schreiber, S. L. Science, 2000, 289, 1760-1763; MacBeath, G.; et al. J. Am. Chem. Soc., 1999, 121, 7967-7968; Falsey, J. R.; et al. Bioconj. Chem., 2001, 12, 346-353; Houseman, B. T.; et al. Nat. Biotechnol., 2002, 20, 270-274; Lesaicherre, M. L.; et al. Bioorg. Med. Chem. Lett. 2002, 12, 2079-2083; Lesaicherre, M. L.; et al. Bioorg. Med. Chem. Lett. 2002, 12, 2085-2088; Uttamchandani, M.; et al. Bioorg. Med. Chem. Lett., 2003, 13, 2997-3000; Oliver, C. et al. Bioconj. Chem., 2003, 14, 430-439; Newman, J. R. S.; Keating, A. E. Science, 2003, 300, 2097-2101; Kiyonaka, S.; et al. Nat. Materials, 2004, 3, 58-64; Li, S.; et al. Am. Chem. Soc., 2004, 126, 4088-4089; Cheng, C. W.; et al. Bioorg. Med. Chem. Lett., 2004, 14, 1987-1990; Houseman, B. T.; et al. Langmuir., 2004, 19, 1522-1531; Lesaicherre, M. L.; Lue et al. J. Am. Chem. Soc. 2002, 124, 8768-8769; Lue, R. Y. P.; et al. J. Am. Chem. Soc., 2004, 126, 1055-1062; Salisbury, C. M. et al. J. Am. Chem. Soc. 2002, 124, 14868-14870; Zhu, Q.; et al. Org. Lett., 2003, 5, 1257-1260; and Soellner, M. B.; et al. J. Am. Chem. Soc., 2003, 125, 11790-11791, each herein incorporated by reference in their entirety for all purposes. Non-limiting illustrative examples include in situ peptide arrays synthesis techniques where unique peptide sequences are simultaneously synthesized on a glass surfaces, or spotting (also sometimes referred to as printing) techniques where presynthesized peptides are spotted onto a suitably derivatized display surface, e.g., a glass surface. Both in situ peptide arrays synthesis techniques and spotting techniques are described in more detail in U.S. Pub. Nos. US20110071043A1 and US20140087963A1, each herein incorporated by reference in their entirety for all purposes. Lateral flow assays are based on ELISA techniques and generally involve attaching and/or spotting antibodies or analytes to a capillary membrane configured such that applying a liquid sample on one end of the capillary membrane will “flow” the samples along the membrane to interact with the analyte and/or antibody. Non-limiting exemplary sandwich-type lateral flow assays are described in greater detail in U.S. Pat. Nos. 4,168,146 and 4,366,241, each of which is herein incorporated by reference in their entirety for all purposes. Other exemplary device configurations and/or assay formats are also described in greater detail in U.S. Pat. Nos. 5,395,754; 5,670,381; and 6,194,220, each of which is herein incorporated by reference in their entirety for all purposes.

The display surfaces (e.g., the solid supports) can also possess further modifications or functionalized properties that aid in the methods and processes of capturing and isolating binding molecules and/or other applications, such as sequencing (e.g., barcodes facilitating NGS applications). As non-limiting illustrations, solid supports can have magnetic properties that allow for magnetic isolation or have fluorescent molecules attached, for example, fluorophores that facilitate fluorescence-activated cell sorting (FACS). Other properties may also include the ability to remove (e.g., elute or cleave off) bound peptides from the display surface, such as reversible linkers (e.g., disulfide reduction of thiol linkers) or protease cleavage sites (e.g., TEV protease recognition motif).

### 6.3. Peptide Expression Libraries

In one aspect, peptide expression library compositions are provided. In a particular aspect, peptide expression library compositions comprising peptides corresponding to epitopes associated with Leptospirosis are provided, as described in further detail below.

Peptide expression library compositions comprise a library of nucleic acid sequences encoding a library of peptides. As used herein, a “library of peptides” or a “peptide library” refers to a collection of a peptide fragments typically used for screening purposes. The terms “peptide,” “polypeptide,” “amino acid sequence,” “peptide sequence,” and “protein” are used interchangeably to refer to two or more amino acids linked together and imply no particular length. Amino acids and peptides can be naturally occurring or synthetic (e.g., non-natural amino acids or amino acid analogs). Amino acids and peptides can also comprise, or be further modified to comprise, reactive groups, such as reactive groups for attaching amino acids or peptides to solid substrates, reactive groups for labeling amino acids or peptides, or reactive groups for attaching other moieties of interest to amino acids or peptides. Reactive groups include, but are not limited to, chemically-reactive groups such as reactive thiols (e.g., maleimide based reactive groups), reactive amines (e.g., N-hydroxysuccinimide based reactive groups), “click chemistry” groups (e.g., reactive alkyne groups), and aldehydes bearing formylglycine (FGly).

In general, a peptide expression library contains a large variety of distinct peptides. For example, the diversity of the library (sometimes referred to as “complexity” of the library) can be at least 10⁴, at least 10⁵, at least 10⁶, at least 10⁷, at least 10⁸, at least 10⁹, at least 10¹⁰, or at least 10¹¹ distinct peptides. The library can be a random peptide expression library where the amino acid sequences are unbiased. A particular embodiment of a random/unbiased library is one constructed to represent all possible amino acid sequences of designated length(s).

A peptide expression library can also be a non-random library where the amino acid sequences are biased in their representation. For example, a library can be biased to represent, over represent, predominantly represent, or only represent amino acid sequences characteristic of a particular feature, such as epitopes or antigens associated with a particular disease (e.g., a bacterial infection, a viral infection, a parasitic infection, an autoimmune disorder, cancer, allergies etc.), condition, species (e.g., mammal, human, bacteria, virus etc.), protein, class of proteins, protein motif (e.g., phosphorylation motifs, binding motifs, protein domains, etc.), amino acid property (e.g., hydrophobic, hydrophilic, acidic, basic, or steric amino acid properties), or any other subset of amino acid sequences that is rationally designed. A library can be biased to also avoid certain amino acid sequences or motifs. A library can be biased to also avoid certain amino acid sequences or motifs. A library can be biased to represent one distinct peptide species of a particular motif, e.g., one distinct peptide species representative of an epitope or epitope motif. For example, a library can have one distinct peptide species representative of peptide motif sequences GPAG[EQ] (SEQ ID NO:29), PA[GA][TD]XW (SEQ ID NO:32), and [AP]AGT[MLI] (SEQ ID NO:33). A library can be biased to represent one distinct peptide species of a particular motif class, e.g., one distinct peptide species representative of motifs that are known or are likely to encompass the same epitope. For example, a library can have one distinct peptide species representative of either peptide motif sequence GPAG[EQ] (SEQ ID NO:29), PA[GA][TD]XW (SEQ ID NO:32), and [AP]AGT[MLI] (SEQ ID NO:33) which can be considered as part of the same motif class. Without wishing to be bound by theory, inclusion of more than one peptide featuring slight variations, e.g., more than one peptide from the same motif class, can yield better enrichments and/or capture slightly different subsets of subjects.

A peptide library can also combine the features of a non-random and random peptide library. For example, one or more select positions within an amino acid sequence may be a constant amino acid and other positions within the sequence may be fully random or biased based on other properties. In other examples, one or more select positions within an amino acid sequence may be selected from a defined subset of amino acids. One skilled in the art will appreciate that the various biases described can combined to achieve a desired purpose of the peptide library, such as a targeted screen.

Typically, peptides in a library can also all fall within a range of lengths. For example, the peptides in a library may be different lengths, but all fall within a defined range of lengths. The selected range can be any length useful for the present invention, such as any length suitable for displaying an epitope sequence capable of recognition by a binding molecule. The peptides in a library can be at least 5, 6, 7, 8, 9, 10, 11, 12, 13, 14, 15, 16, 17, 18, 19, or 20 amino acids in length. The peptides in a library can also be 5-30, 5-25, 5-20, 5-15, 5-10, 10-30, 10-25, 10-20, or 10-15 amino acids in length. The peptides in a library can also be 7-14, 8-14, 9-14, 10-14, 11-14, 12-14, 7-13, 8-13, 9-13, 10-13, 11-13, 12-13, 7-12, 8-12, 9-12, 10-12, 11-12, 7-11, 8-11, 9-11, or amino acids in length. If desired, the peptides in the library can also be greater than 30, greater than 40, greater than 50, greater than 75, greater than 100, greater than 200, or greater than 300 amino acids in length.

Peptides in a library can also be an identical defined length, i.e., all the peptides in the library have the same number of amino acids. The defined length can be any length useful for the present invention, such as any length suitable for displaying an epitope sequence capable of recognition by a binding molecule. The defined length can be 5, 6, 7, 8, 9, 10, 11, 12, 13, 14, 15, 16, 17, 18, 19, or 20 amino acids in length.

The nucleic acid sequences in the peptide expression library can be constructed to achieve a desired library property including those described above, such as peptide diversity, peptide randomization or biasing, and/or peptide length. For example, nucleic acid sequences in the peptide expression library can be biased for expression in specific organisms, such as codon optimization for expression in bacterial, viral, plant, yeast/fungal, insect, mammalian, or human expression systems. Any suitable nucleic acid allowing expression of the peptides of interest may be used. In general, the nucleic acid will be a vector. As used herein, a “vector” refers to nucleic acid construct capable of directing the expression of a gene of interest, typically in a host organism, such as a bacterial cell, a mammalian cell (e.g., a human cell), a bacteriophage, a yeast cell, an insect cell, or a plant cell. A vector typically contains the appropriate transcriptional and translational regulatory nucleotide sequences recognized by the desired host for peptide expression, such as promoter sequences. A promoter sequence can be a constitutive promoter. A promoter sequence can be an inducible promoter, where transcription of the encoded sequences is induced by addition of an analyte, chemical, or other molecule, such as a Tet-on system. A variation of an inducible promoter system is a system where transcription is actively repressed, and addition of an analyte, chemical, or other molecule removes the repression, such as addition of arabinose for an arabinose operon promoter or a Tet-off system. A vector can also include elements that facilitate vector construction and production, such as restriction sites, sequences that direct vector replication, drug selection genes or other selectable markers, and any other elements useful for cloning and library production. A typical vector can be a double stranded DNA plasmid in which the nucleic acid sequences encoding the desired peptides is inserted using standard cloning techniques in a location and orientation capable of directing peptide expression. Other vectors include, but are not limited to, nucleic acid constructs useful for in vitro transcription and translation, linear nucleic acid constructs, and single-stranded DNA or RNA nucleic acid constructs.

In general, the number of copies of a specific nucleic acid sequence for each of the candidate peptides is present at a roughly equivalent number, though some variation in number may occur due to probability. A typical peptide expression library can contain more than one copy of a specific nucleic acid sequence (e.g., multiple copies of the same vector). However, in examples where a plurality of samples each contain members of the peptide expression library, the absolute number of each of the candidate peptides may not be equivalent between samples. For example, zero or one copy of a specific nucleic acid sequence can be present in a given sample while one or more copies may be present in another given sample. While the number of copies of a specific nucleic acid sequence need not be identical to the number of copies of other specific nucleic acid sequences, it is generally assumed that about the same number of sequences are present for each of the candidate peptides.

Peptide expression libraries include, but are not limited to, bacterial expression libraries, yeast expression libraries, bacteriophage expression libraries, ribosome expression libraries, and mammalian expression libraries. Particular peptide libraries and peptide expression libraries useful for the present invention are described in more detail in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference in their entirety for all purposes.

### 6.3.1. Arrayed Peptide Expression Libraries

In a series of embodiments, a composition is composed of two or more of the peptide expression library compositions described above. The two or more peptide expression library compositions can each be contained in a separate container, such as a well in a multi-well plate, a microcentrifuge tube, a test tube, a tube, and a PCR tube. Each of the separate containers can comprise the same library of nucleic acid sequences encoding the library of peptides or a collection of different peptide expression libraries. The collection of peptide expression library compositions can be 2, 3, 4, 5, 6, 7, 8, 9, 10-15, 16-24, 24-48, 48-96, or 96-384 peptide expression library compositions. The collection of peptide expression library compositions can be at least 10, at least 20, at least 50, at least 100, at least 200, at least 300, at least 500, at least 1000, or at least 2000 expression library compositions.

### 6.4. Binding Molecules

As used herein, “binding molecules” refer to those molecules that specifically and selectively bind a binding target(s). In a typical non-limiting example, a binding molecule is an antibody. An antibody can be a monoclonal antibody, which are typically produced from cultured antibody-producing cell lines, or a polyclonal antibody, which are typically produced by collecting the antibody containing serum of an animal immunized with the antigen or epitope of interest, or fragment thereof. Antibodies can also refer to antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In other examples, the binding molecule(s) can refer to binding molecules other than antibodies including, but not limited to, aptamers, peptoids, and affibodies.

A binding molecule (e.g., an antibody) or antigen binding portion thereof is said to “recognize” the epitope (or more generally, the antigen) to which the binding molecule specifically binds, and the epitope (or more generally, the antigen) is said to be the “recognition specificity” or “binding specificity” of the binding molecule.

A binding molecule is said to bind to its specific antigen or epitope with a particular affinity. As described herein, “affinity” refers to the strength of interaction of non-covalent intermolecular forces between one molecule and another. The affinity, i.e., the strength of the interaction, can be expressed as a dissociation equilibrium constant (KD), wherein a lower KD value refers to a stronger interaction between molecules. “Specific binding,” as used herein, refers to an affinity between an ABS and its cognate antigen or epitope in which the KD value is below 10⁶M, 10⁻⁷M, 10⁻⁸M, 10⁻⁹M, or 10⁻¹⁰M. KD values of binding molecules (e.g. antibodies) are measured by methods well known in the art including, but not limited to, bio-layer interferometry (e.g., Octet/FORTEBIO®), surface plasmon resonance (SPR) technology (e.g., Biacore®), and cell binding assays.

### 6.5. Biological Samples

As used herein, a “biological sample” refers to any material known to contain or suspected to contain specimen binding molecules (e.g., antibodies). In general, the sample will be a liquid. The sample can be a material that originated as a liquid or can be material processed to be in liquid form. The sample can be the material directly isolated from a source (i.e., untreated) or it can be further processed for use in the method (e.g., diluted, filtered, cell depleted, particulate depleted, assayed, preserved, or other otherwise pre-processed). Samples include, but are not limited to, serum, blood, saliva, urine, tissue, tissue homogenates, stool, spinal fluid, and lysate derived from animal sources. The sample can include a mixture of different source materials. A sample can be a bodily fluid isolated from any animal that produces or suspected to produce the binding molecule of interest. The animal can be known or suspected of having a disease. The animal can also be known or suspected of having binding molecules that bind antigens or epitopes associated with the disease. In an illustrative non-limiting example, the sample can be processed serum from a human suspected to have a specific disease and suspected to produce antibodies that bind epitopes that correlate with the disease. Diseases include, but are not limited to, a bacterial infection, a viral infection, a parasitic infection, an autoimmune disorder, cancer, and an allergy.

### 6.6. Capture Entities

In one example of the invention, the display surfaces, binding molecules, and/or peptides described above are bound to or capable of being bound to a capture entity. As used herein, a “capture entity” refers to any entity useful for capturing or isolating a display surface, a binding molecule, and/or a peptide. The binding molecule can be any of the binding molecules described herein, such as a binding molecule in a biological sample (e.g., serum antibodies in a patient's sample). The display surface can be any of the display surfaces described herein, such as a display surface in a peptide display system (e.g., a solid support or biological entity).

In a typical example, the capture entity comprises a solid support. Solid supports are described in greater detail in Section 6.2.3 titled “Display surfaces.”

The capture entities (e.g., the solid supports) can also be modified or functionalized to bind and capture (i.e., immobilize) display surfaces, binding molecules, and/or peptides. Such modifications can be attaching to or coating the surface of a capture entity with a “binding moiety” that binds the display surfaces, binding molecules, and/or peptides. Examples include binding moieties that specifically bind antibodies, such as Protein A, Protein G, Protein A/G, Protein L, or an anti-immunoglobulin antibody. Other examples include chemical modifications that allow formation of covalent bonds with proteins (e.g., activated aldehyde groups) and modifications that facilitate specific pairing via cognate modifications (e.g., biotin-streptavidin pairs, disulfide linkages, polyhistidine-nickel, or “click-chemistry” modifications such as azido-alkynyl pairs). As a non-limiting illustration, a binding molecule can be modified to contain a biotin moiety (i.e., biotinylated) and a capture entity can be modified to contain streptavidin on its surface. Capture entities can also possess further modifications or functionalized properties that aid in the methods and processes of capturing and isolating display surfaces, binding molecules, and/or peptides. As non-limiting illustrations, capture entities can have magnetic properties that allow for magnetic isolation, or capture entities can have fluorescent molecules attached that allow, for example, fluorescence-activated cell sorting (FACS). Other properties include the ability to remove (e.g., elute or cleave off) captured display surfaces, binding molecules (and/or their respective binding target), and/or peptides if desired.

In a non-limiting example, in the methods describe herein where a binding molecule is used to bind a binding target, the binding molecule can either be immobilized on a capture entity prior to the binding molecule binding its target (e.g., prior to contacting or mixing the peptide library with the binding molecule), or the binding molecule can be immobilized on a capture entity subsequent to the binding molecule binding its target. Similarly, display surfaces and/or peptides can either be immobilized on a capture entity prior to contacting another reagent, such as a sample, or subsequent to contacting another reagent.

As a non-limiting illustration of a capture entity, a magnetic bead is functionalized with Protein A/G (e.g., Pierce). The Protein A/G bead is subsequently used to capture antibodies bound to their respective epitope targets.

### 6.7. Methods and Assays Associated with Leptospirosis Epitope Motifs

In particular aspects, any of the peptide display systems or peptide expression libraries described herein can be used in an assay. Examples of types of assays the peptide display systems or peptide expression libraries described herein are useful for include, but are not limited to, binding assays (e.g., antibody binding assays), diagnostic assays, vaccine efficacy and safety studies, immune status monitoring assays. In particular, provided for herein are assays, such as binding assays (e.g., antibody binding assays), diagnostic assays, vaccine efficacy and safety studies, immune status monitoring assays, using peptide display systems or peptide expression libraries containing epitopes associated with Leptospirosis. Accordingly, the peptide display systems or peptide expression libraries described herein can be formulated for use in an assay method, such as any of the assays described herein, including, but not limited to, a method of determining the presence of specimen binding molecules (e.g., antibodies) specific for Leptospirosis, a method of diagnosing a subject for Leptospirosis, a method of treating a subject known or suspected of suffering from Leptospirosis, a method of determining whether a subject is a candidate for treatment of Leptospirosis, and/or a method of determining the binding affinity of specimen binding molecules (e.g., antibodies) for an epitope associated with Leptospirosis.

In one aspect, a method of determining the presence of specimen binding molecules (e.g., antibodies) specific for Leptospirosis in a biological sample is provided. The method of determining the presence of specimen binding molecules (e.g., antibodies) specific for Leptospirosis in a biological sample includes: contacting any of the display systems and/or peptide expression libraries described herein with a biological sample (e.g., a biological sample from a subject), wherein the biological sample contains a plurality of binding molecules (e.g., antibodies), wherein the plurality of binding molecules is known or suspected to contain specimen binding molecules specific for Leptospirosis, and wherein the contacting is under conditions sufficient for the specimen binding molecules specific for Leptospirosis to bind a cognate epitope; measuring the binding between the distinct peptides of the display systems and/or peptide expression libraries and the specimen antibodies; and determining specimen binding molecules specific for Leptospirosis are present in the biological sample when binding between at least one of the at least two distinct peptides and the specimen binding molecules is detected. The assay can determine the presence of binding molecules (e.g., antibodies) that bind multiple distinct peptides. The assay can determine the presence of binding molecules (e.g., antibodies) that bind at least one of the multiple distinct peptides. The assay can determine the presence of binding molecules (e.g., antibodies) that bind at least 2, 3, 4, 5, 6, 7, 8, 9, 10 or more distinct peptides of the multiple distinct peptides. The assay can determine the presence of binding molecules (e.g., antibodies) that bind at least one of the at least one distinct peptides. The assay can determine the presence of binding molecules (e.g., antibodies) that bind the at least two distinct peptides. The assay can determine the presence of binding molecules (e.g., antibodies) that bind at least one of the at least two distinct peptides. The assay can determine the presence of binding molecules (e.g., antibodies) that bind 3, 4, 5, 6, 7, 8, 9, 10 or more distinct peptides. The assay can determine the presence of binding molecules (e.g., antibodies) that bind at least one of the 3, 4, 5, 6, 7, 8, 9, 10 or more distinct peptides. The assay can determine the presence of binding molecules (e.g., antibodies) that bind each of the one or more distinct peptides on the array. The assay can determine the presence of binding molecules (e.g., antibodies) that bind a motif encompassing the one or more distinct peptides on the array. The assay can determine the presence of binding molecules (e.g., antibodies) that bind a motif class encompassing the one or more distinct peptides on the array. The binding molecules that bind the multiple distinct peptides can be multiple distinct binding molecules that bind the multiple distinct peptides. The binding molecules that bind the multiple distinct peptides can include a single distinct binding molecule that binds two or more of the multiple distinct peptides, i.e., include a cross-reactive binding molecule. Determining the presence can also include determining the quantity or relative quantity (e.g., concentration) of a distinct binding molecule for a distinct peptide. Determining the presence can also include determining the quantity or relative quantity (e.g., concentration) of binding molecules for a peptide motif.

In one aspect, a method of diagnosing Leptospirosis in a subject is provided. The method of diagnosing Leptospirosis in a subject includes: contacting any of the display systems and/or peptide expression libraries described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen binding molecules specific for Leptospirosis to bind a cognate epitope; measuring the binding between the distinct peptides of the display systems and/or peptide expression libraries and the specimen antibodies. The subject can be diagnosed as: i) positive for Leptospirosis when binding between at least one of the at least one distinct peptides and the specimen antibodies is detected, or ii) negative for Leptospirosis when binding between less than one of the at least one distinct peptides and the specimen antibodies is detected (e.g., none). The subject can be diagnosed as: i) positive for the Leptospirosis infection when binding between at least 2, at least 3, at least 4, or at least 5 distinct peptides of the at least one distinct peptides and the specimen antibodies is detected, or ii) negative for the Leptospirosis infection when binding between less than at least 2, at least 3, at least 4, or at least 5 distinct peptides, respectively, of the at least one distinct peptides and the specimen antibodies is detected. The subject can be diagnosed as: i) positive for the Leptospirosis infection when binding between at least 6, at least 7, at least 8, at least 9, or at least 10 distinct peptides of the at least one distinct peptides and the specimen antibodies is detected, or ii) negative for the Leptospirosis infection when binding between less than at least 6, at least 7, at least 8, at least 9, or at least 10 distinct peptides, respectively, of the at least one distinct peptides and the specimen antibodies is detected. The subject can be diagnosed as: i) positive for the Leptospirosis infection when a z-score meets or exceeds a threshold value, or ii) negative for the Leptospirosis infection when a z-score is less than the threshold value. A z-score for each motif can be calculated as the enrichment value minus the mean enrichment for all samples divided by the standard deviation of all samples. A z-score can be a value calculated for a single motif. A z-score can be a composite z-score value (also referred to herein as a summed z-score, diagnostic score, or composite diagnostic score) in which the z-scores for each motif are summed for a specific disease.

In one aspect, a method of treating a subject known or suspected of suffering from Leptospirosis is provided. The method of treating the subject includes: assessing or having assessed whether the subject suffers from Leptospirosis, wherein the assessment includes contacting any of the display systems and/or peptide expression libraries described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen binding molecules specific for Leptospirosis to bind a cognate epitope; measuring the binding between the distinct peptides of the display systems and/or peptide expression libraries and the specimen antibodies; assessing the subject; and administering the therapy when the subject is assessed as positive. The subject can be assessed as: i) positive for Leptospirosis when binding between at least one of the at least one distinct peptides and the specimen antibodies is detected, or ii) negative for Leptospirosis when binding between less than one of the at least one distinct peptides and the specimen antibodies is detected (e.g., none). The subject can be assessed as: i) positive for the Leptospirosis infection when binding between at least 2, at least 3, at least 4, or at least 5 distinct peptides of the at least one distinct peptides and the specimen antibodies is detected, or ii) negative for the Leptospirosis infection when binding between less than at least 2, at least 3, at least 4, or at least 5 distinct peptides, respectively, of the at least one distinct peptides and the specimen antibodies is detected. The subject can be assessed as: i) positive for the Leptospirosis infection when binding between at least 6, at least 7, at least 8, at least 9, or at least 10 distinct peptides of the at least one distinct peptides and the specimen antibodies is detected, or ii) negative for the Leptospirosis infection when binding between less than at least 6, at least 7, at least 8, at least 9, or at least 10 distinct peptides, respectively, of the at least one distinct peptides and the specimen antibodies is detected. The subject can be assess as: i) positive for the Leptospirosis infection when a z-score meets or exceeds a threshold value, or ii) negative for the Leptospirosis infection when a z-score is less than the threshold value. A z-score can be a value calculated for a single motif. A z-score can be a composite z-score value. Therapies for treating Leptospirosis can be any of method known to those skilled in the art and may include, but are not limited to, antibiotics, such as doxycycline or penicillin, and in more severe cases intravenous antibiotics.

In one aspect, a method of determining whether a subject is a candidate for treatment of Leptospirosis is provided. The method of treating the subject includes: assessing or having assessed whether the subject suffers from Leptospirosis, wherein the assessment includes contacting any of the display systems and/or peptide expression libraries described herein with a biological sample from the subject, wherein the biological sample comprises a plurality of antibodies, wherein the plurality of antibodies is known or suspected to comprise specimen antibodies specific for Leptospirosis, and wherein the contacting is under conditions sufficient for the specimen binding molecules specific for Leptospirosis to bind a cognate epitope; measuring the binding between the distinct peptides of the display systems and/or peptide expression libraries and the specimen antibodies; assessing the subject; and determining the subject is a candidate for a treatment of Leptospirosis when the subject is assessed as positive. The subject can be assessed as: i) positive for Leptospirosis when binding between at least one of the at least one distinct peptides and the specimen antibodies is detected, or ii) negative for Leptospirosis when binding between less than one of the at least one distinct peptides and the specimen antibodies is detected (e.g., none). The subject can be assessed as: i) positive for the Leptospirosis infection when binding between at least 2, at least 3, at least 4, or at least 5 distinct peptides of the at least one distinct peptides and the specimen antibodies is detected, or ii) negative for the Leptospirosis infection when binding between less than at least 2, at least 3, at least 4, or at least 5 distinct peptides, respectively, of the at least one distinct peptides and the specimen antibodies is detected. The subject can be assessed as: i) positive for the Leptospirosis infection when binding between at least 6, at least 7, at least 8, at least 9, or at least 10 distinct peptides of the at least one distinct peptides and the specimen antibodies is detected, or ii) negative for the Leptospirosis infection when binding between less than at least 6, at least 7, at least 8, at least 9, or at least 10 distinct peptides, respectively, of the at least one distinct peptides and the specimen antibodies is detected. The subject can be assess as: i) positive for the Leptospirosis infection when a z-score meets or exceeds a threshold value, or ii) negative for the Leptospirosis infection when a z-score is less than the threshold value. A z-score can be a value calculated for a single motif. A z-score can be a composite z-score value. Therapies for treating Leptospirosis can be any of method known to those skilled in the art and may include, but are not limited to, antibiotics, such as doxycycline or penicillin, and in more severe cases intravenous antibiotics.

In one aspect, a method of determining the binding affinity of specimen binding molecules (e.g., antibodies) for an epitope associated with Leptospirosis in a biological sample is provided. The method of determining the binding affinity includes: contacting the display system and/or peptide expression library with a biological sample from the subject, wherein the biological sample contains a plurality of binding molecules (e.g., antibodies), wherein the plurality of binding molecules is known or suspected to contain specimen binding molecules specific for Leptospirosis, and wherein the contacting is under conditions sufficient for the specimen binding molecules specific for Leptospirosis to bind a cognate epitope; and measuring the binding affinity between the at least two distinct peptides and the specimen binding molecules. Measuring the binding affinity can include determining if a binding molecule specifically binds an epitope associated with Leptospirosis.

As used herein, “contacting” refers to any method of bringing the specimen binding molecules in proximity to and under conditions sufficient for binding to their respective binding targets, e.g., the distinct peptides in the display system and/or protein expression library. The contacting of the different components can be performed in any suitable order. Providing suitable conditions for a binding molecule (e.g., an antibody) to bind a respective binding target (e.g., epitope or epitope motif) can include controlling conditions, including but not limited to, temperature, pH, salt, buffer, binding molecule concentration, and/or target concentration.

Contacting can include mixing all the compositions together. Mixing can be performed in a container, such as a well in a multi-well plate, a microcentrifuge tube, a test tube, a tube, and a PCR tube. Mixing can include rotating, incubating, pipetting, inverting, vortexing, shaking, or otherwise mechanically disturbing components.

“Isolating” steps used herein can be any method useful for retrieving specimen and control binding molecules. Isolation can involve the use of capture entities, described in more detail in Section 6.6. Isolation methods include, but are not limited to magnetic isolation, bead centrifugation, resin centrifugation, and FACS. A particular isolation method can be selected based on the properties of a capture entity, if used, for example magnetic isolation of magnetic beads or FACS isolation of fluorescent beads.

“Measuring” steps, as used herein, in general can use any method for detecting binding between a binding molecule and its cognate epitope, such as optical scanning, an ELISA, fluorescent detection, digital serology, flow-cytometry, surface plasmon resonance, bio-layer interferometry, lateral flow assay, or combinations thereof. In a particular non-limiting example, the E. coli expression library described herein can be used in conjunction with digital serology to measure binding. The binding measurements can be quantitative, such as determining binding affinity. The binding measurements can be qualitative, such as determining a binary result of whether a binding molecule does or does not bind a distinct peptide (e.g., determining whether the measured binding exceeds a threshold for the binding molecule to be considered to bind or specifically bind a distinct peptide).

Nucleic acid sequence determination steps, as used herein, in general can use any method for sequencing and/or quantifying nucleic acid, such next generation sequencing (NGS) or quantitative polymerase chain reaction (qPCR). Examples of NGS technologies include massively parallel sequencing techniques and platforms, such as Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies. In some embodiments, the determining step contains the steps of 1) purifying the nucleotide from the biological entity; 2) amplifying the purified nucleic acid sequences encoding a peptide bound by the isolated specimen binding molecules; and 2) sequencing the amplified nucleotides. The nucleic acid to be sequenced can also be further modified or processed to facilitate sequencing. For example, nucleic acid can be modified for multiplexed high-throughput sequencing of multiple samples simultaneously, such as adding a sample identifying nucleic acid sequence unique to the sample to terminus of the amplified nucleotides during the amplification step.

Various nucleic acid sequences (e.g., sequences encoding a library of peptides) can be differentiated from each other during the sequence determining step(s). Differentiating various nucleic acid sequences includes differentiating portions of nucleic acid sequences, such as differentiating the different sequences in a vector (e.g., differentiating a nucleic acid sequence encoding a binding target from other vector nucleic acid sequences). Sequences can be differentiated based on specific characteristics, such as position within a sequence, identity of adjacent sequences, known identity of sequences, or combinations thereof. Sequence alignment algorithms, such as those known in the art, can be used to identify, quantify, and differentiate the different sequences.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder described herein. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment),

As used herein, the term “administering,” refers to the placement an agent as disclosed herein into a subject by a method or route which results in at least partial localization of the agents at a desired site.

### 6.7.1. Computer

Many of the assays described herein (e.g., motif analysis, sequence alignment/clustering, NGS applications, etc.) typically require the use of a computer as they cannot be practically carried out by the human mind or by pen and paper alone. In general, a computer is adapted to execute a computer program for providing results, for example the results of determining nucleic acid sequences such as those sequences produced during a sequencing step or the results of an assessment step providing if the assay meets a quality control standard. Generally, the steps of determining the nucleic acid sequences and determining the results of the assessment step involve such a large number of computations, particularly given the number of sequences generally under consideration, that they are carried out by a computer system in order to be completed in a reasonable amount of time. They cannot be practically carried out by the human mind or by pen and paper alone. A computer can include at least one processor coupled to a chipset. Also coupled to the chipset can be a memory device, a memory controller hub, an input/output (I/O) controller hub, and/or a graphics adaptor. Various embodiments of the invention may be implemented as a computer program instructions stored in a non-transitory computer readable storage medium for execution by a processor of a computer system. The instructions define functions of the embodiments (including the methods described herein). Illustrative computer-readable storage media include, but are not limited to: (i) non-writable storage media (e.g., read-only memory devices within a computer such as CD-ROM disks readable by a CD-ROM drive, flash memory, ROM chips or any type of solid-state non-volatile semiconductor memory) on which information is permanently stored; and (ii) writable storage media (e.g., floppy disks within a diskette drive or hard-disk drive or any type of solid-state random-access semiconductor memory) on which alterable information is stored. A computer can include a means for programming the computer (i.e., providing computer program instructions), such as providing sequence alignment software or quality control assessment software. A computer can include a means for inputting information, such as sequences, including, but not limited to, a keyboard, a mouse, a touch-screen interface, or combinations thereof. A computer can include a means to display information and images, such as a graphics adaptor and display. A computer can include means to connect to other computers (e.g., computer networks), such as a network adaptor. Some portions of the description herein describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs, equivalent electrical circuits, or the like. The described operations and their associated modules may be embodied in software, firmware, hardware, or any combinations thereof. A quality control standard can be a ratio or percentage of unique peptide sequences specific present in a sample. The ratio or percentage can be relative to unique peptide sequences that are not specific to a sample (e.g., present as a result of sample contamination). The ratio or percentage can also be relative to the total number of any subset of nucleic acid sequences useful for establishing a quality control standard, such as sequences encoding a library of peptides, sequences encoding a control binding target, unique nucleic acid sequences, or combinations thereof.

A computer, as described herein, can be used to perform determination (e.g., sequencing) and assessment steps described herein.

The different methods described herein are not mutually exclusive.

### 6.8. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### 6.8.1. Digital Serology Method

### 6.8.1.1. Overview

Digital Serology is a Next-generation Sequencing (NGS)-based assay similar to other biopanning assays in which peptide libraries are screened with human serum to map human antibody repertoires. The assay involves 4 main steps: 1) incubation of serum with the peptide library and affinity selection of library members expressing peptides that are specific to the antibody repertoire for each serum sample; 2) purification of plasmids that encode these peptides; 3) PCR amplification of the region of the plasmids encoding the peptides (amplicons) and barcoding of each sample with sample-specific primers (allowing samples to be pooled and sequenced together on a single NGS run); and 4) amplicon sequencing by NGS. Once the amplicons are sequenced, the data are demultiplexed and antibodies present in each serum sample are identified based on the peptide motifs to which they bind.

### 6.8.1.2. Methods

The methods described below are useful for the examples described herein but are not intended to be limiting. Methods useful for the present invention, e.g., digital serology including motif determination and motif analysis, are also described in more detail in Pantazes, et al. and international PCT application WO2017083874A1, each herein incorporated by reference in their entirety for all purposes.

### Serum Collection

Sixty paired acute and convalescent serum samples from thirty patients hospitalized in Brazil for confirmed Leptospira interrogans by Microscopic Agglutination Test (MAT), PCR and/or culture, as well as endemic control serum that was negative for Leptospirosis by MAT (“Endemic Controls”) were provided by Albert Ko, Professor of Epidemiology (Microbial Diseases) and of Medicine (Infectious Diseases), Yale University. Healthy serum samples (n=306) from the database obtained from commercial sources were used in discovery to normalize individual motif enrichment scores. To evaluate panel specificity, the Leptospirosis IgG and IgM panels were run on 1500 database samples from individuals tested for acute illness. Leptospirosis testing was not performed on these samples, thus the reported SERA specificity is the lower limit for specificity. Given the sensitivity limitations of MAT, it is possible that some of these were misclassified by MAT.

### Bacterial Surface Display Antibody Screen

A large, high-quality, bacterial-display, random, 12-mer peptide library composed of 8×10⁹ independent transformants, was constructed using trinucleotide oligos to eliminate stop codons and normalize amino acid usage frequencies. The 12-mer peptide library was displayed on E. coli via the N-terminus of a previously reported, engineered protein scaffold (eCPX), as described in more detail in Rice, et al., herein incorporated by reference for all it teaches. Vectors, methods, and other tools useful in the E. coli surface displayed peptide library are described in more detail in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference for all they teach.

The bacterial display peptide library was used to screen and isolate peptide binders to antibodies in individual serum samples through Magnetic Activated Cell Sorting (MACS). The MACS screen employed magnetic selection to enrich the library for antibody binding peptides as well as reduce the library size suitable for the subsequent screening steps. A frozen aliquot of the library containing 8×10¹⁰ cells (10-fold the diversity of the 12-mer X12 library) was thawed and inoculated into inoculated into multiple 2L baffled flasks of 500 mL LB (10 g/L tryptone, 5 g/L yeast extract, and 10 g/L NaCl) supplemented with 34 μg/mL chloramphenicol. The flasks were incubated with vigorous shaking (300 rpm) at 37° C. until the cells grew to OD₆₀₀=1.0. Peptide expression was induced with L-(+)-arabinose at a final concentration of 0.02% wt/vol for one hour. Cells were collected by centrifugation (3500 rcf for 30 minutes) and resuspended in cold phosphate buffered saline with 15% glycerol (PBSG). The cell suspension was divided into wells of a 96-deep-well plate to yield 10-fold the X12 diversity per well (8×10¹⁰ cells), frozen and stored at −80° C.

Prior to incubation with serum, cells were cleared of peptides that bind protein A/G by incubating cells with washed protein A/G magnetic beads (Pierce) at a ratio of one bead per 50 cells for 45 min. at 4° C. with gentle mixing. Magnetic separation for 5 min. (×2) was used to recover the unbound cells. Recovered cells from the supernatant are centrifuged, resuspended in diluted sera (1:25) and incubated for a one-hour primary incubation at 4° C. with orbital shaking (800 rpm). Following serum incubation, cells were then collected by centrifugation (3500 rcf for 7 minutes), the supernatant was removed, and the cell pellets were washed by resuspending fully in 750 uL PBS+0.05% Tween-20 (PBST). The cells were again collected by centrifugation (3500 rcf for 7 minutes) and the PBST supernatant was removed. Cell pellets were resuspended in 750 uL PBS and mixed thoroughly with 50 uL Protein A/G Sera-Mag SpeedBeads (GE Life Sciences, 17152104010350) (6.25% the beads' stock concentration) were used to identify IgG motifs. The plate was incubated for one hour at 4° C. with orbital shaking (800 rpm). Bead-bound cells were captured in the plate using a Magnum FLX 96-ring magnet (Alpaqua, A000400) until all beads were separated. Unbound cells in the supernatant were removed by gentle pipetting so as not to disturb the beads, leaving only those cells bound to A/G beads. To exclude residual unbound cells and wash non-antibody interactions of cells to beads, the beads were washed by removing from the magnet, resuspending in 750 uL PBST, and then returning to the magnet. The supernatant was removed by gentle pipetting after the beads were securely captured. Beads were washed 5×, and cells were resuspended in 750 uL LB with 34 μg/mL chloramphenicol and 0.2% wt/vol glucose directly in the 96-deep-well plate and grown overnight with shaking (300 rpm) at 37° C.

The following modifications were used to identify IgM motifs. In place of protein A/G beads, a biotinylated monoclonal antibody specific for human IgM (Jackson Immunoresearch, 709-066-073) was added to the mixture of library cells and serum, followed by cell capture on 100 ul Dynabeads MyOne streptavidin T1 coated magnetic beads (ThermoFisher Scientific, 65601).

### Next Generation Sequencing

After growth, cells were collected by centrifugation (3500 rcf for 10 minutes) and the supernatant was discarded. Plasmids were isolated in 96-well format using the Montage Plasmid Miniprep_{HTS} Kit (MilliPore, LSKP09604) on a Multiscreen_{HTS} Vacuum Manifold (MilliPore, MSVMHTS00) following the “Plasmid DNA-Full Lysate” protocol in the product literature. For amplicon preparation, two rounds of PCR were employed; the first round amplifies the variable “X12” peptide region of the plasmid DNA. The second round barcodes each patient amplicon library with sample-specific indexing primers for data demultiplexing after sequencing. KAPA HiFi HotStart ReadyMix (KAPA Biosystems, KK2612) was used as the polymerase master mix for all PCR steps. Plasmids (2.5 uL/well) were used as template for a first round PCR with 12.5 uL of KAPA ReadyMix and 5 uL each of 1 uM forward and reverse primers. The primers (Integrated DNA Technologies) contain annealing regions that flank the X12 sequence (indicated in bold) and adapter regions specific to the Illumina index primers used in the second round PCR.

Forward Primer (SEQ ID NO: 1):
TCGTCGGCAGCGTCAGATGTGTATAAGAGACAGVBHDVCCAGTCTGGCC
AGGG
Reverse Primer (SEQ ID NO: 2):
GTCTCGTGGGCTCGGAGATGTGTATAAGAGACAGGTGATGCCGTAGTAC
TGG

A series of five degenerate bases in the forward primer, VBHDV (following IUPAC codes and italicized in the forward primer sequence above), provided base diversity for the first five reads of the sequencing on the NextSeq platform. The five base pairs were designed to be non-complementary to the template to avoid bias during primer annealing. To reduce non-specific products, a touchdown PCR protocol was used with an initial annealing temperature of 72° C. with a decrease of 0.5° C. per cycle for 14 cycles, followed by 10 cycles with annealing at 65° C. The 25 uL primary PCR product was purified using 30 uL Mag-Bind TotalPure NGS Beads (Omega Bio-Tek, M1378-02) according to the manufacturer's protocol. The second round PCR (8 cycles, 70° C. annealing temperature) was performed using Nextera XT index primers (Illumina, FC-131-2001) which introduce 8 base pair indices on the 5′ and 3′ termini of the amplicon for data demultiplexing of each sample screened. The PCR 1 product (5 uL) was used as a template for the second PCR with 5 uL each of forward and reverse indexing primers, 5 uL PCR grade water and 25 uL of KAPA ReadyMix. The PCR product (50 uL) was cleaned up with 56 uL Omega Mag-Bind TotalPure NGS Beads per reaction. A 96-well quantitation was performed using the Qubit dsDNA High Sensitivity assay (Invitrogen, Q32851) adapted for a microplate fluorimeter (Tecan SPECTRAFlour Plus) measuring fluorescence excitation at 485 nm and emission at 535 nm. Positive (100 ng) and negative (Ong) controls, included with the Qubit kit, were added to the plate as standards along with 2 uL of each PCR product diluted 1:100 for quantitation. The fluorescence data were used to calculate DNA concentration in each well based on the kit standards. To normalize the DNA and achieve equal loading of each patient sample on NGS, the DNA in each well was diluted with Tris HCl (pH 8.5, 10 mM) to 4 nM and an equal volume from each well was pooled in a Lo-Bind DNA tube for sequencing.

The sample pool was prepared for sequencing according to specifications of the Illumina NextSeq 500. Due to the low diversity in the adapter regions of the amplicon after the first five bases, PhiX Run Control (Illumina, FC-110-3001) was included at 40% of the final DNA pool. The pool was sequenced using a High Output v2, 75 cycle kit (Illumina, FC-404-2005).

### Disease-Specific Motif Discovery

Following NGS, the computational algorithm IMUNE was utilized to analyze the set of peptides (i.e., epitope repertoire) identified by sequencing and discover candidate Leptospirosis-specific peptide motifs. IMUNE compared 30 Leptospirosis positive repertoires with 30 control repertoires. IMUNE identified patterns, defined as three to five amino acids interspersed with undefined residues (“X”) within a 10-amino acid window that were significantly enriched in disease samples but not enriched in controls. The enrichment of a pattern was quantified as the ratio of pattern observations in a sample to the expected observations taking into account the amino acid frequencies in the peptide library and total number of sequences for that sample. Patterns were selected if they were statistically significant (p<0.0001) in the specified percentage of disease samples and not statistically significant (p>0.5/# of controls) in the specified percentage of control samples.

To generate epitope motifs, significantly enriched patterns (˜10⁴-10⁵ patterns) were aligned and scored using a PAM30 substitution matrix as described previously. Unlike patterns, motifs may contain positions where multiple, often similar, amino acids are observed, indicated by brackets (e.g., [IVL] or [KRQ]. Pattern clustering generated ˜100s of motifs. Enrichments for motifs were calculated as the number of observations of a specific motif within a repertoire/the number expected by random chance.

### Phenotype Determination and Disease Diagnosis

Following NGS analysis, samples were analyzed for enrichment for each motif in a panel of peptide motifs that were previously identified as diagnostic motifs for specific diseases. The enrichment was calculated by dividing the number of observed instances for each motif by the number of expected instances. A z-score for each motif was calculated where each z-score indicates the enrichment value minus the mean enrichment for all samples divided by the standard deviation of all samples. To calculate a composite diagnostic score, the z-scores for each motif were summed for the specific disease. Diagnostic thresholds, e.g., whether a sample was considered to be positive for a disease, were established for each condition balancing sensitivity and specificity.

### Microscopic Agglutination Test (MAT)

MAT tests were performed in accordance with the literature (Goris M G and Hartskeerl R A. Leptospirosis serodiagnosis by the microscopic agglutination test. Curr Protoc Microbiol. 2014 Feb. 6; 32:Unit 12E.5, herein incorporated by reference for all purposes).

Determination of Motifs Associated with Leptospirosis

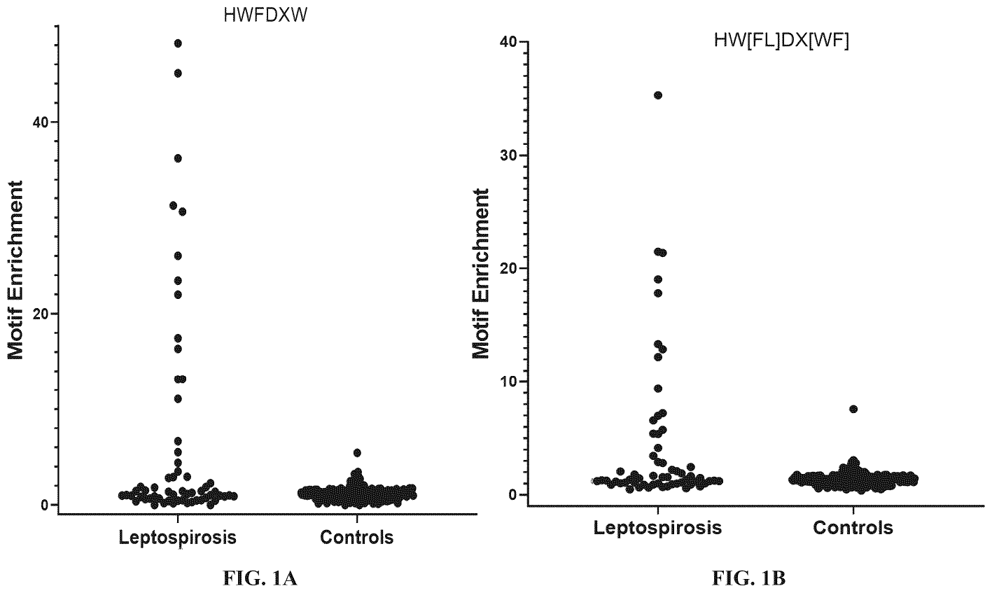

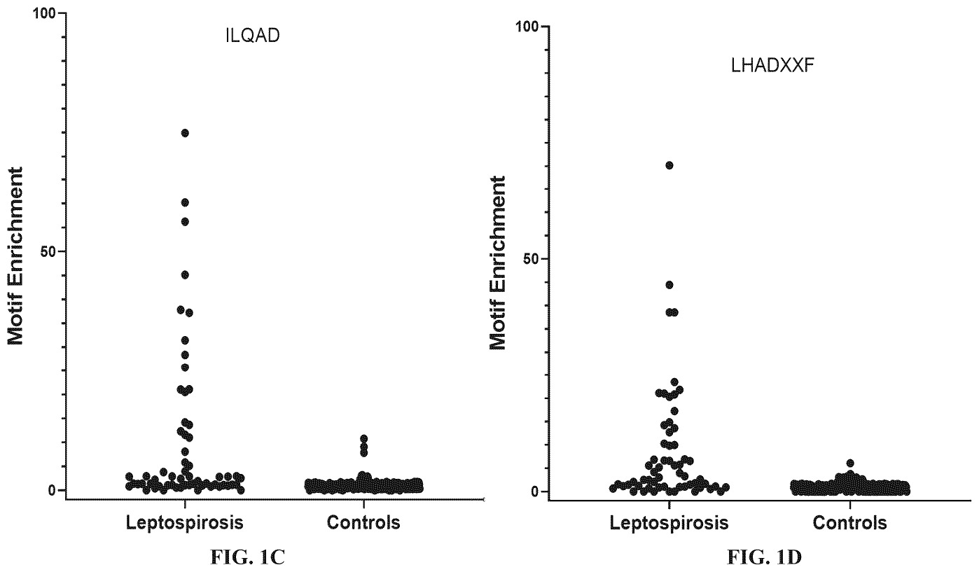

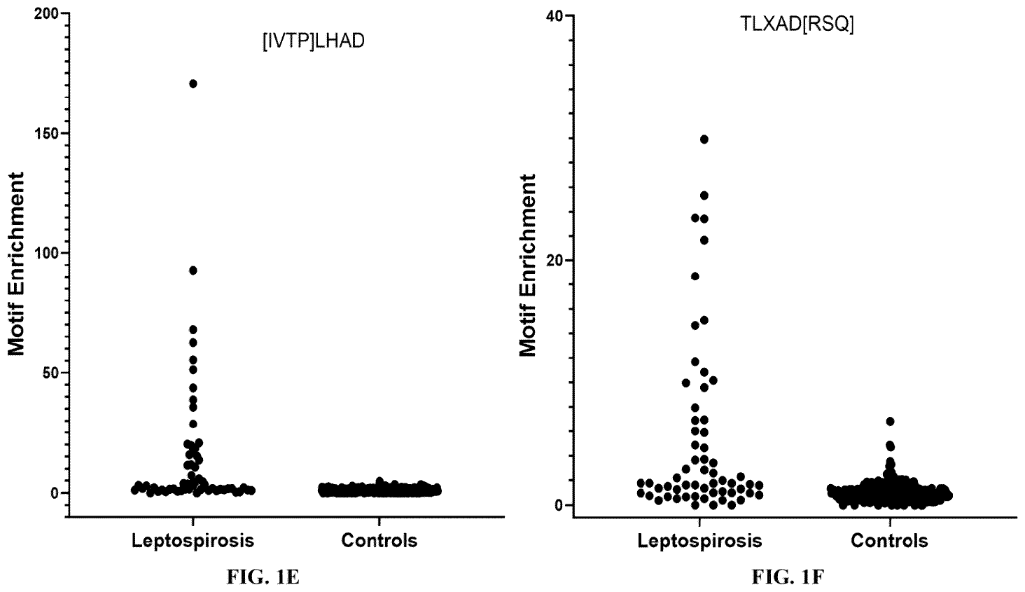

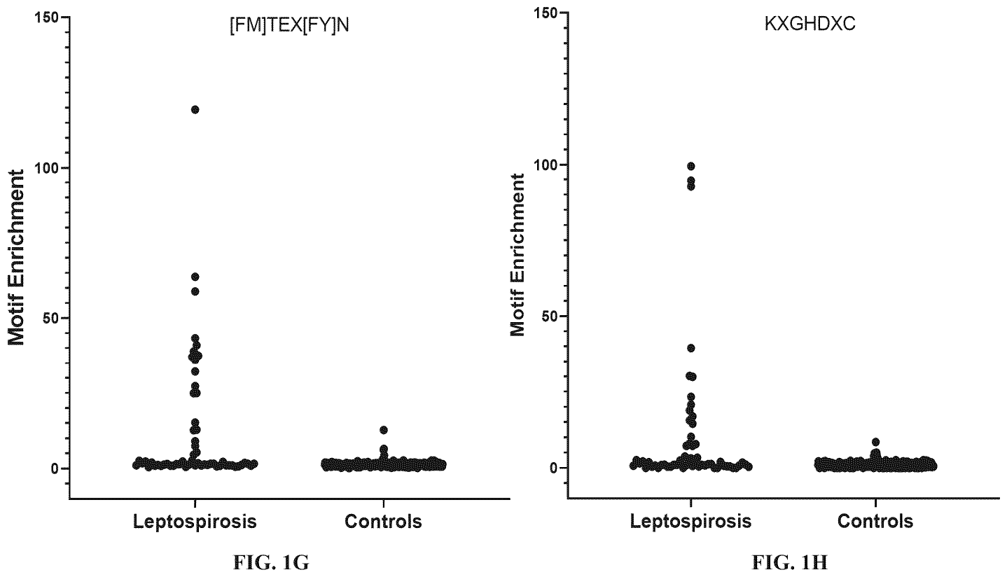

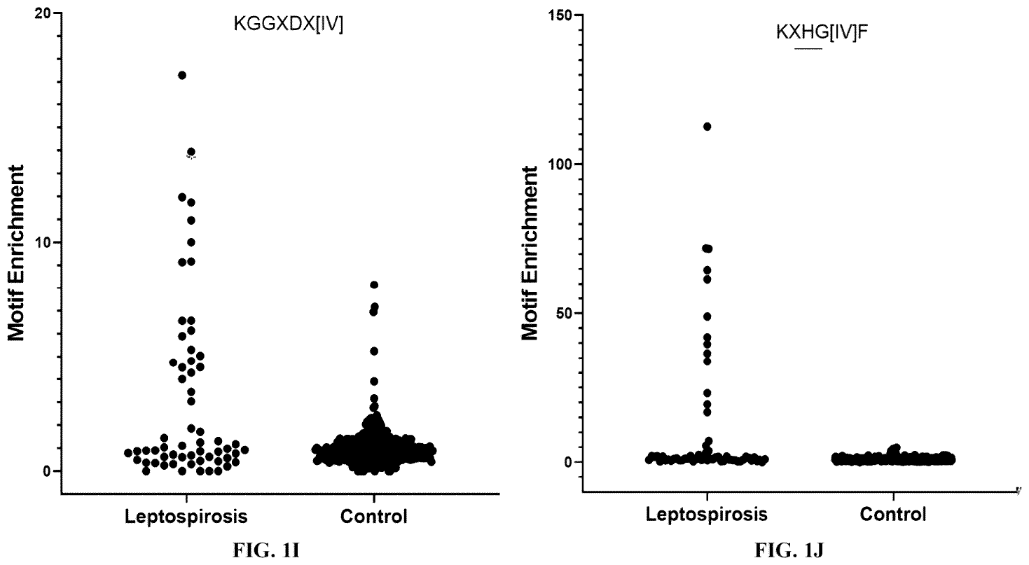

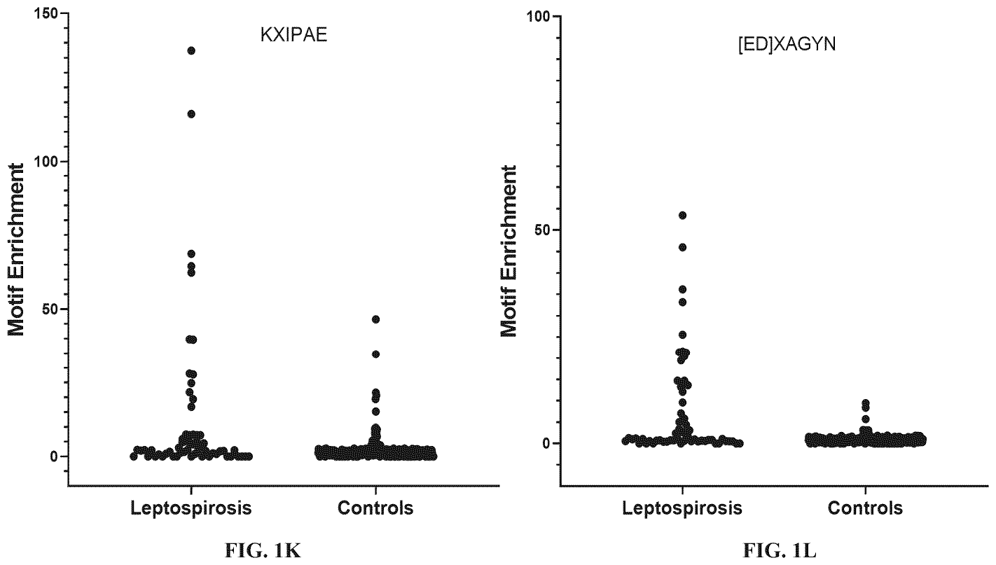

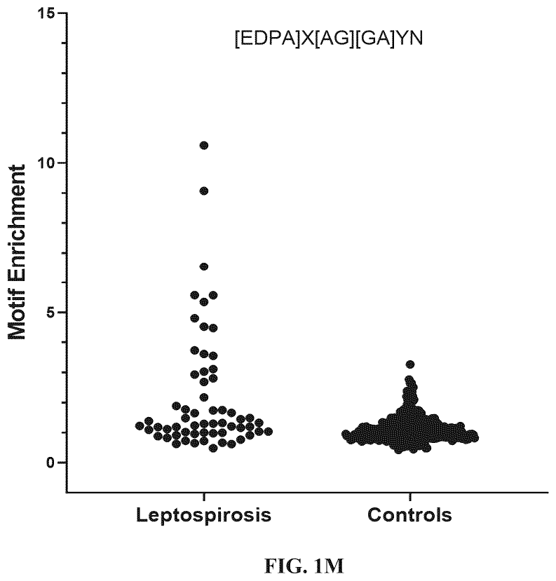

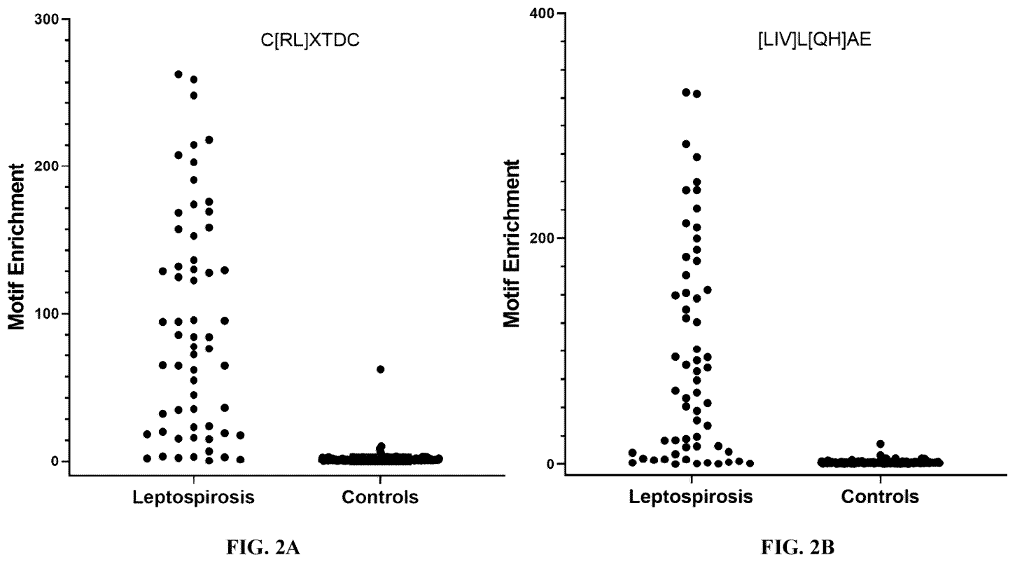

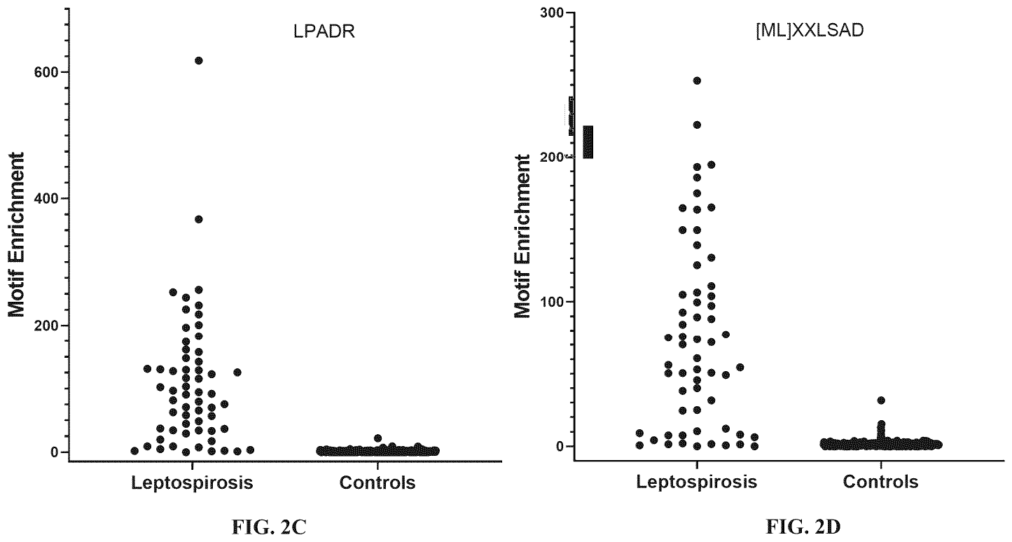

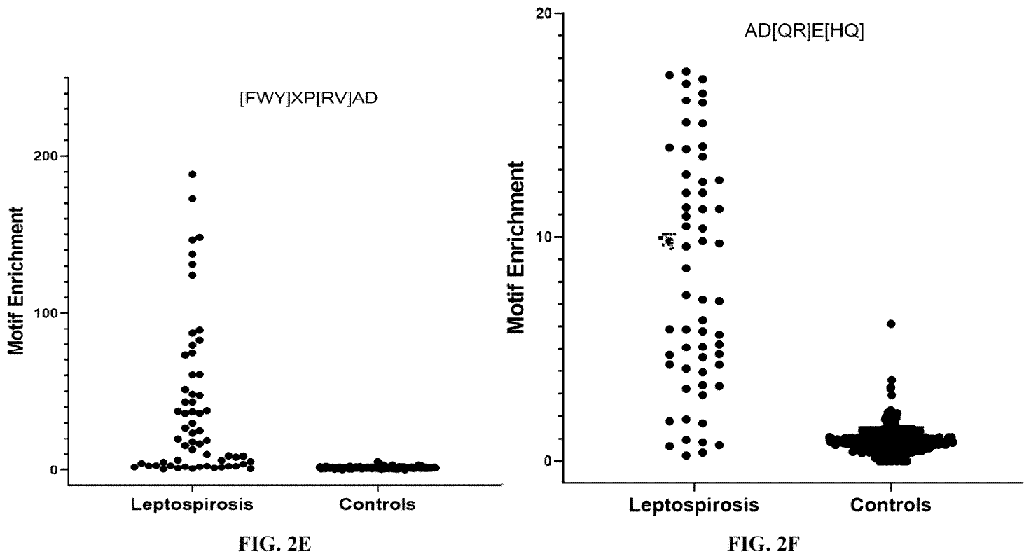

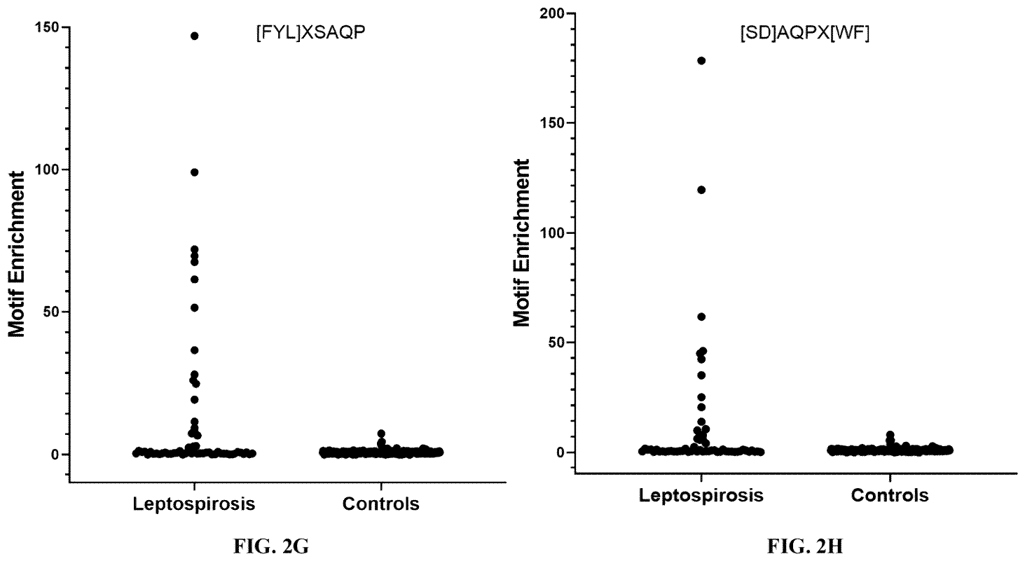

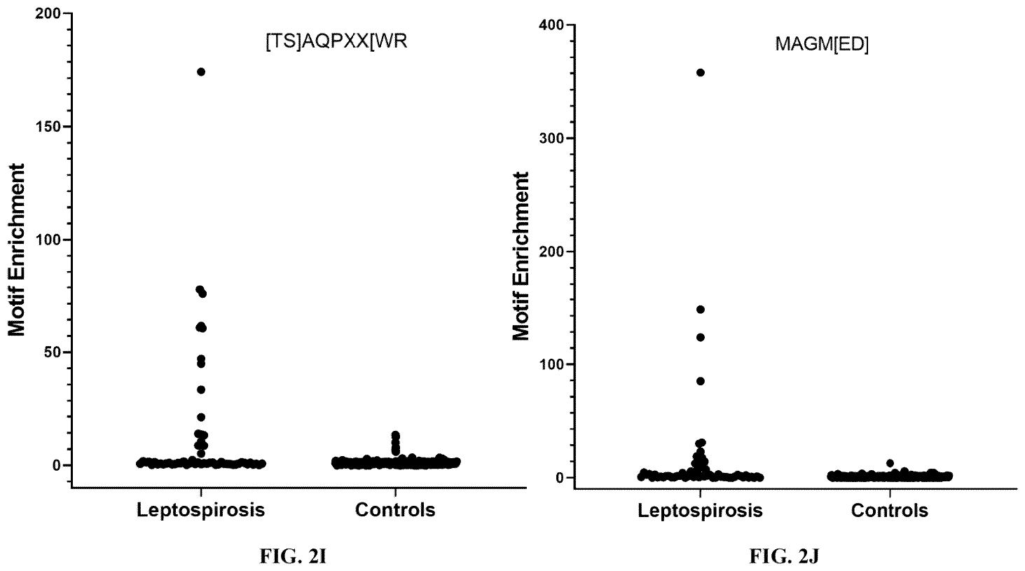

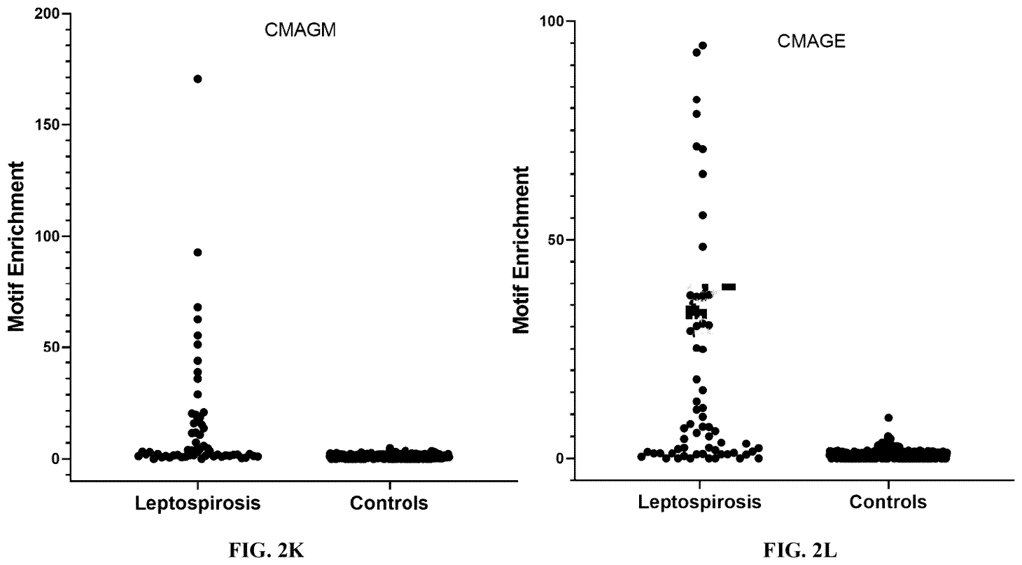

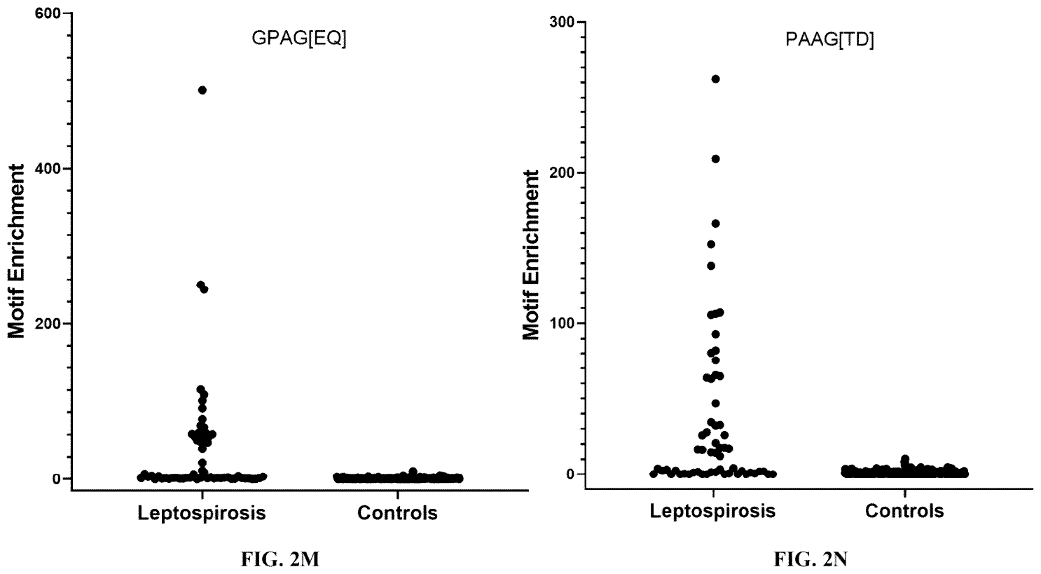

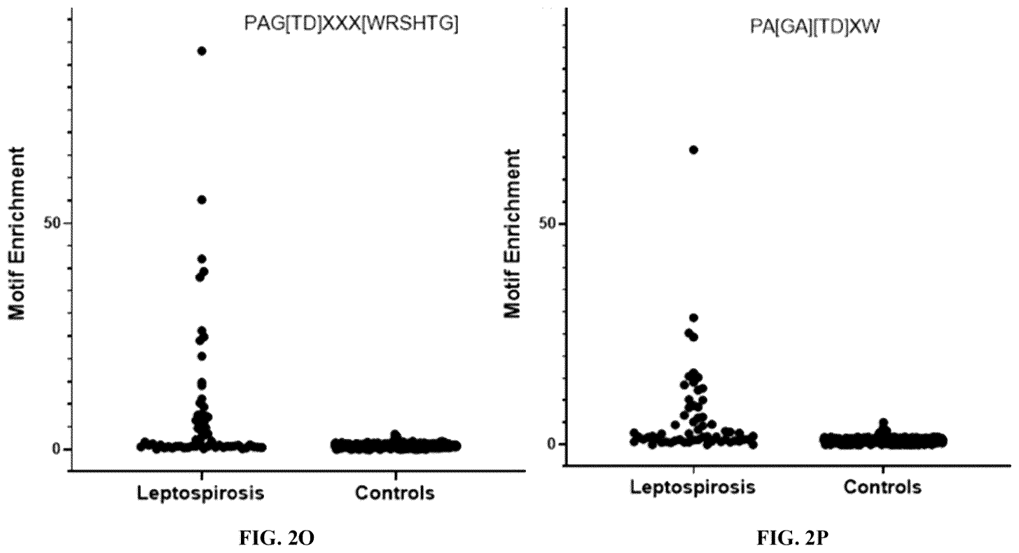

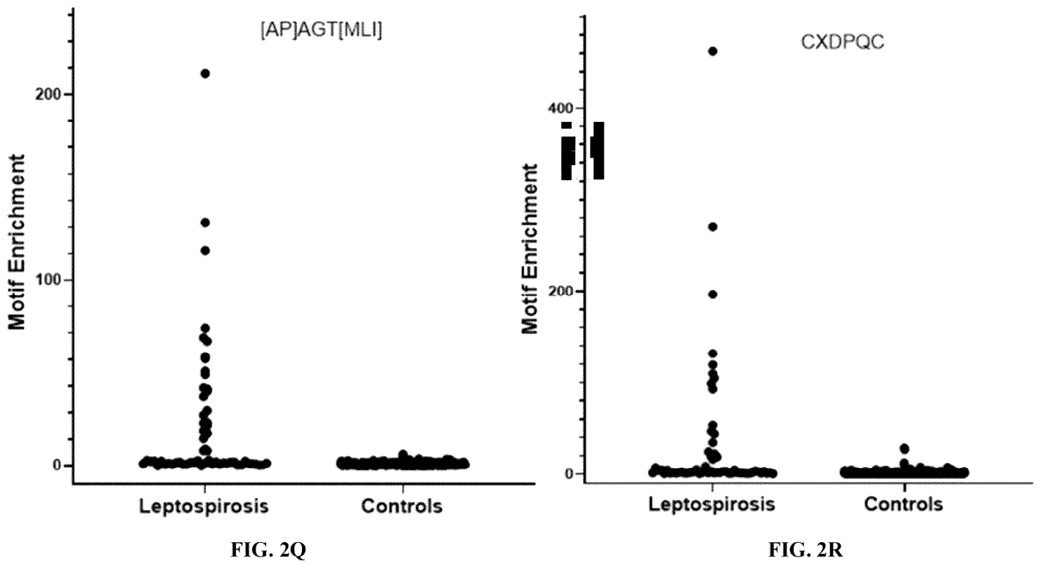

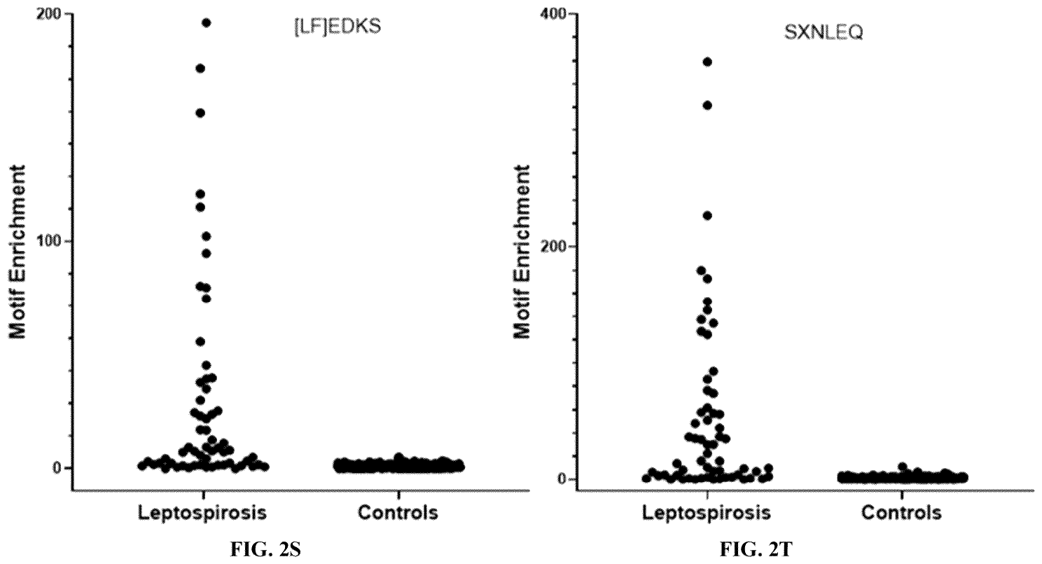

Antibody binding motifs for Leptospirosis were determined using the SERA digital serology, as described above. IgG and IgM epitope motif panels are presented in Tables 1 and 2 below, respectively. Each of the final IgG and IgM panel epitope motifs were enriched in ≥20% of disease and <2% of ˜300 control repertoires, where a positive enrichment is defined as four standard deviations above the mean of the control group. Enrichments for the motifs in each panel are shown in FIG. 1A-M (IgG) and FIG. 2A-T (IgM). Individual motifs ranged from an average of 5-fold to 200-fold enrichment over the control group and each motif was present in a unique subset of patients.

TABLE 1
Leptospira IgG Epitope Motifs

| | SEQ | | |
|---|---|---|---|
| | ID | Discovery | Discovery |
| IgG Motif | NO: | Sensitivity | Specificity |
| [FM]TEX[FY]N | 4 | 33% | 99% |
| KXGHDXC | 5 | 28% | 99% |
| KGGXDX[IV] | 6 | 27% | 99% |
| KXHG[IV]F | 7 | 25% | 99% |
| HWFDXW | 8 | 27% | 100% |
| HW[FL]DX[WF] | 9 | 27% | 100% |
| ILQAD | 10 | 33% | 99% |
| LHADXXF | 11 | 35% | 100% |
| [IVTP]LHAD | 12 | 65% | 99% |
| TLXAD[RSQ] | 13 | 32% | 99% |
| KXIPAE | 14 | 20% | 98% |
| [ED]XAGYN | 15 | 33% | 99% |
| [EDPA]X[AG][GA]YN | 16 | 25% | 100% |

TABLE 2
Leptospira IgM Epitope Motifs

| | SEQ | | |
|---|---|---|---|
| | ID | Discovery | Discovery |
| IgM Motif | NO: | Sensitivity | Specificity |
| C[RL]XTDC | 17 | 83% | 99.7% |
| [LIV]L[QH]AE | 18 | 80% | 99.4% |
| LPADR | 19 | 85% | 99.0% |
| [ML]XXLSAD | 20 | 75% | 99.0% |
| [FWY]XP[RV]AD | 21 | 73% | 99.4% |
| AD[QR]E[HQ] | 22 | 80% | 99.4% |
| [FYL]XSAQP | 23 | 28% | 99.0% |
| [SD]AQPX[WF] | 24 | 30% | 98.7% |
| [TS]AQPXX[WR] | 25 | 27% | 98.4% |
| MAGM[ED] | 26 | 28% | 99.7% |
| CMAGM | 27 | 42% | 100.0% |
| CMAGE | 28 | 50% | 100.0% |
| GPAG[EQ] | 29 | 45% | 99.7% |
| PAAG[TD] | 30 | 53% | 99.0% |
| PAG[TD]XXX[WRSHTG] | 31 | 43% | 99.4% |
| PA[GA][TD]XW | 32 | 38% | 99.7% |
| [AP]AGT[MLI] | 33 | 42% | 99.4% |
| CXDPQC | 34 | 30% | 99.0% |
| [LF]EDKS | 35 | 62% | 99.7% |
| SXNLEQ | 36 | 67% | 99.4% |

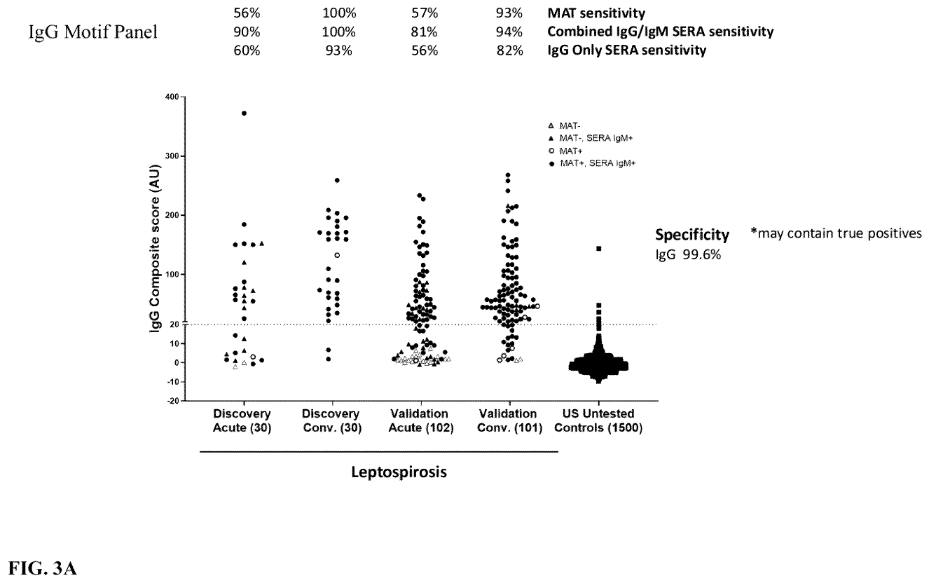

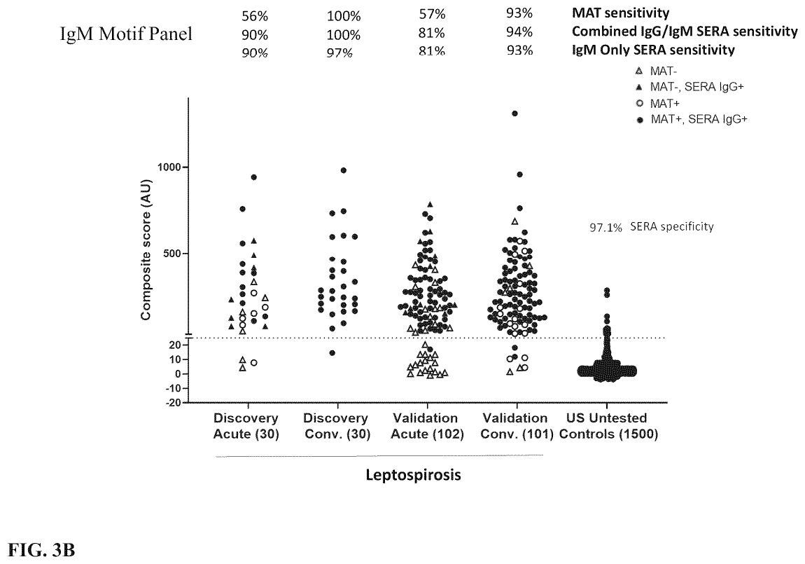

Motif enrichments were converted to z-scores and summed to generate a composite diagnostic score. A cut off was chosen that yielded 100% specificity on the discovery control set and taking into consideration the natural separation within the Leptospirosis positive repertoires. Seventeen of thirty acute discovery Leptospirosis patient sera were MAT positive for Leptospira interrogans serovar L1-130 (n=13) or other serovars (n=4) for an overall MAT sensitivity on the acute discovery blood draw of 56% with a similar sensitivity of 57% for the acute validation sera panel. All thirty of the convalescent discovery panel sera (100%) and 93% of the convalescent validation panel sera were MAT positive. The Leptospirosis SERA IgG only panel (FIG. 3A) was 60% and 56% sensitive (similar to MAT) for the acute discovery and validation blood draws, respectively, as well as 93% and 82% sensitive for the convalescent discovery and validation blood draws, respectively. The SERA IgM only panel (FIG. 3B) was 90% and 81% sensitive (greater than MAT) for the acute discovery and validation blood draws, respectively, as well as 97% and 93% sensitive (similar to MAT) for the convalescent discovery and validation blood draws, respectively. Notably, combining analysis of both IgG and IgM panels achieved an overall sensitivity of 90% and 81% (greater than MAT) for the acute discovery and validation blood draws, respectively, as well as 100% and 94% (equivalent to MAT) for the convalescent discovery and validation blood draws, respectively.

To evaluate panel specificity, Leptospirosis IgG and IgM scores were first determined for 1500 database samples from individuals tested for acute illness by the SERA panels (notably, prior Leptospirosis testing was not performed on these samples, thus the reported SERA specificity is considered the lower limit for specificity). Specificity by the IgG SERA panel was determined to be 99.6% (again, the lower range given true positives potentially present), by the IgM SERA panel to be 97.1%, and by the combined IgG/IgM panel to be 96.6%. An additional 193 samples that were collected from endemic regions and for which negative MAT results were available (as assessed by IgG), for which the specificity of the SERA IgG panel for this cohort was determined to be 97.9% and by the SERA IgM panel determined to be 96.3% (data not shown).

In summary, peptide motifs have been determined that serve as diagnostic biomarkers of Leptospirosis infection, in particular in assessing acute infection, with superior sensitivity and specificity relative to existing gold standard MAT testing.

### 6.8.2. ELISA/LFA-Based Diagnostic Assay

Peptide sequences were selected for inclusion in ELISA and LFA diagnostic assay formats. Motifs were selected based on the combination that yielded the highest combined sensitivity and specificity criteria. Peptides (12-mers) were selected from motifs that either (1) mapped onto a native antigen encoded by Leptospira interrogans serovar L1-130, or (2) if no direct equivalent to a native peptide was determined, by querying Leptospirosis patient repertoires that were positive for the motif and identifying the most enriched peptide (the peptide that appeared in the greatest number of subjects).

The selected peptides are incorporated into ELISA and LFA display formats, including as concatenated peptides, and assessed.

TABLE 3
Motifs and Peptides for ELISA/LFA Formats

| Motif | Antigen | Peptide | Isotype |
|---|---|---|---|
| LHADXXF (SEQ ID NO: 11) | LipL45 | EAKILHADLTEK (SEQ | IgG and M |
| | | ID NO: 37) | |
| HWFDXW(SEQ ID NO: 8) | LipL32 | SNPHWFDTWIRV (SEQ | IgG |
| | | ID NO: 38) | |
| | OMPL1 | APRKAIPAENRL (SEQ | IgG |
| | | ID NO: 39) | |
| | | TKADIAGYNIVD (SEQ | IgG |
| | | ID NO: 40) | |
| | | NIKGGYDILTIA (SEQ | IgG |
| | | ID NO: 41) | |
| (PAG and MAG family) | no direct | HQGWAAGTIVVG (SEQ | IgM |
| MAGM[ED] (SEQ ID NO: 26), | equivalent to | ID NO: 42) | |
| CMAGM (SEQ ID NO: 27), | native | VWGLGPAGQWDT (SEQ | IgM |
| CMAGE (SEQ ID NO: 28) | peptide | ID NO: 43) | |
| GPAG[EQ] (SEQ ID NO: 29); | | | |
| PA[GA][TD]XW (SEQ ID | | | |
| NO: 32); PAAG[TD] (SEQ ID | | | |
| NO: 30); [AP]AGT[MLI] | | | |
| (SEQ ID NO: 33) | | | |
| KXHG[IV]F | no direct | KWPGIFSMPPMD (SEQ | IgG |
| (SEQ ID NO: 7) | equivalent to | ID NO: 44) | |
| | native | RSQKQQGIFSFA (SEQ | |
| | peptide | ID NO: 45) | |
| | | KHFGIFSEDSWQ (SEQ | |
| | | ID NO: 46) | |
| [FM]TEX[FY]N | no direct | HYTEYFNYDPRK (SEQ | IgG |
| (SEQ ID NO: 4) | equivalent to | ID NO: 47) | |
| | native | GGRWQYTEDFN (SEQ | |
| | peptide | ID NO: 48) | |
| | | ALQDWHFTEQYN (SEQ | |
| | | ID NO: 49) | |

### 6.8.3. Selection of Motifs for Optimal Sensitivity

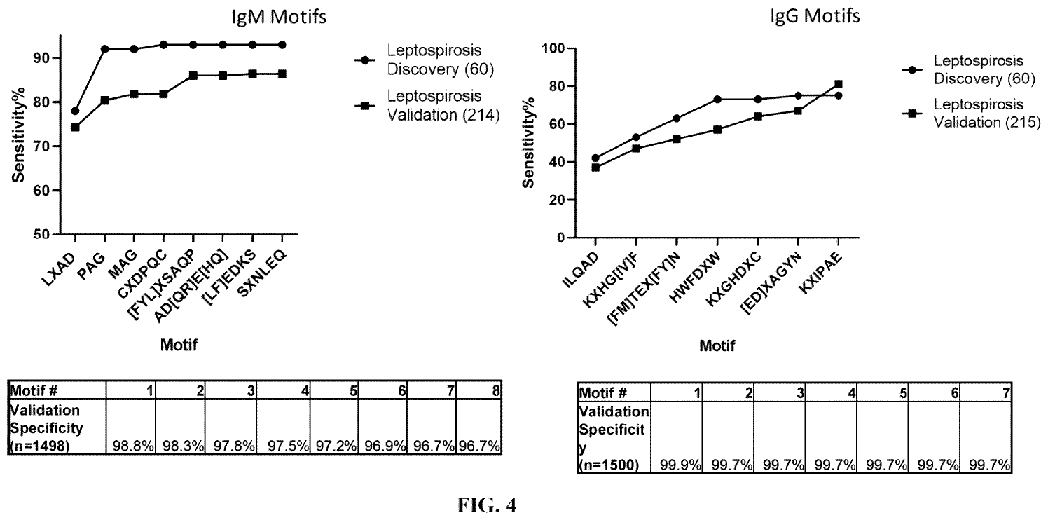

To identify sets of IgG and IgM Leptospirosis-specific epitope motifs that achieve a balance between maintaining specificity while optimizing sensitivity, an algorithm was used to combine determined motifs or motifs representative of a motif-class sequentially, summing the z-scores of each motif/motif-class for each subject at each iteration to maximize the sensitivity in the Leptospirosis cohort while maintaining the specificity at or above 99% for IgG and above 96% for IgM. The motif panel performance was trained on a set of 305 controls and tested on a set of 1500 controls. Iterative performance analysis is presented in FIG. 4 for IgM (left panel) and IgG (right panel) motif/motif-classes and demonstrate analysis of combined motifs improved sensitivity performance while maintaining specificity.

### 7. EQUIVALENTS

While the invention has been particularly shown and described with reference to a preferred embodiment and various alternate embodiments, it will be understood by persons skilled in the relevant art that various changes in form and details can be made therein without departing from the spirit and scope of the invention.

### 8. INCORPORATION BY REFERENCE

All references, issued patents and patent applications cited within the body of the instant specification are hereby incorporated by reference in their entirety, for all purposes.

### 9. REFERENCES CITED

Endrullat C, Glökler J, Franke P, Frohme M. (2016) Standardization and quality management in next-generation sequencing. Appl Transl Genom. 10:2-9.
Rice, J. J. & Daugherty, P. S. Directed evolution of a biterminal bacterial display scaffold enhances the display of diverse peptides. Protein Eng. Des. Sel. 21, 435-442 (2008).
Pantazes, R. J. et al. Identification of disease-specific motifs in the antibody specificity repertoire via next-generation sequencing. Sci. Rep. 6, 30312; doi: 10.1038/srep30312 (2016).
Dayhoff, M. & Schwartz, R. A Model of Evolutionary Change in Proteins. Atlas protein Seq. Struct. 345-352 doi: 10.1.1.145.4315 (1978).

1. A peptide display system, wherein the display system comprises:
(a) one or more display surfaces; and
(b) at least one distinct peptide, wherein the at least one distinct peptides comprises a binding motif, wherein the binding motif is selected from a motif group consisting of: [FM]TEX[FY]N (SEQ ID NO:4), KXGHDXC (SEQ ID NO:5), KGGXDX[IV] (SEQ ID NO:6), KXHG[IV]F (SEQ ID NO:7), HWFDXW (SEQ ID NO:8), HW[FL]DX[WF] (SEQ ID NO:9), ILQAD (SEQ ID NO: 10), LHADXXF (SEQ ID NO:11), [IVTP]LHAD (SEQ ID NO: 12), TLXAD[RSQ] (SEQ ID NO:13), KXIPAE (SEQ ID NO:14), [ED]XAGYN (SEQ ID NO: 15), [EDPA]X[AG][GA]YN (SEQ ID NO: 16), C[RL]XTDC (SEQ ID NO:17), [LIV]L[QH]AE (SEQ ID NO: 18), LPADR (SEQ ID NO: 19), [ML]XXLSAD (SEQ ID NO:20), [FWY]XP[RV]AD (SEQ ID NO:21), AD[QR]E[HQ] (SEQ ID NO:22), [FYL]XSAQP (SEQ ID NO:23), [SD]AQPX[WF] (SEQ ID NO:24), [TS]AQPXX[WR] (SEQ ID NO:25), MAGM[ED] (SEQ ID NO:26), CMAGM (SEQ ID NO:27), CMAGE (SEQ ID NO:28), GPAG[EQ] (SEQ ID NO:29), PAAG[TD] (SEQ ID NO:30), PAG[TD]XXX[WRSHTG] (SEQ ID NO:31), PA[GA][TD]XW (SEQ ID NO:32), [AP]AGT[MLI] (SEQ ID NO:33), CXDPQC (SEQ ID NO:34), [LF]EDKS (SEQ ID NO:35), and SXNLEQ (SEQ ID NO:36), and wherein the at least one distinct peptide extends from the one or more display surfaces.

2. The display system of claim 1, wherein the display system comprises at least 2 distinct peptides each comprising a distinct binding motif, wherein each distinct binding motif is selected from the motif group.

3. The display system of claim 1, wherein the display system comprises at least 3, at least 4, at least 5, at least 6, at least 7, at least 8, at least 9, at least 10, at least 15, at least 20, at least 25, or at least 30 distinct peptides each comprising a distinct binding motif, wherein each distinct binding motif is selected from the motif group.

4. (canceled)

5. (canceled)

6. The display system of claim 1, wherein the at least one distinct peptide is capable of binding to an antibody associated with a Leptospirosis infection.

7. The display system of claim 1, wherein the one or more display surfaces comprises a single display surface.

8. (canceled)

9. (canceled)

10. The display system of claim 7, wherein the single display surface is a peptide microarray, a multi-well plate, or a lateral flow assay.

11. (canceled)

12. (canceled)

13. (canceled)

14. (canceled)

15. (canceled)

16. The display system of claim 6, wherein the one or more display surfaces comprises a plurality of display surfaces.

17. (canceled)

18. (canceled)

19. The display system of claim 16, wherein the plurality of display surfaces is a plurality of biological entity surfaces, and wherein the at least one distinct peptide is encoded by one or more distinct nucleic acid sequences capable of expressing the at least one distinct peptide in the plurality of biological entities.

20. (canceled)

21. (canceled)

22. (canceled)

23. The display system of claim 16, wherein the plurality of display surfaces is a plurality of microparticle surfaces.

24. The display system of claim 16, wherein each of the plurality of display surfaces comprises a distinct single species of the at least one distinct peptides.

25. The display system of claim 1, wherein the at least one distinct peptide further comprises a label.

26. The display system of claim 1, wherein the one or more display surfaces or the distinct peptides is bound or capable of being bound directly or indirectly to a capture entity.

27. The display system of claim 26, wherein the capture entity comprises:
(a) a solid support selected from the group consisting of: an agarose bead, a sepharose bead, a magnetic bead, and a resin; and
(b) either (i) a binding moiety that binds directly to the one or more display surfaces or the distinct peptides or (ii) a binding moiety that binds a binding molecule that binds to the one or more display surfaces or at least one of the distinct peptides.

28. (canceled)

29. (canceled)

30. The display system of claim 27, wherein:
(a) the binding molecule that binds to at least one of the distinct peptides is an antibody from a biological sample; or
(b) either (i) a binding moiety that binds directly to the one or more display surfaces or the distinct peptides or (ii) a binding moiety that binds a binding molecule that binds to the one or more display surfaces or at least one of the distinct peptides; or
(c) the binding moiety comprises streptavidin and the one or more display surfaces, the distinct peptide, or the binding molecule that binds to the one or more display surfaces or at least one of the distinct peptides further comprises biotin.

31. (canceled)

32. (canceled)

33. (canceled)

34. (canceled)

35. (canceled)

36. (canceled)

37. (canceled)

38. The display system of claim 1, wherein the composition further comprises a library of peptides.

39. (canceled)

40. The display system of claim 38, wherein the library of peptides is a non-random peptide library.

41. The display system of claim 40, wherein the non-random library is biased to represent one or more diseases or conditions, wherein the biased representation is at least greater than a representation in a random peptide library, and wherein at least one of the one or more diseases or conditions is a Leptospirosis infection.

42. (canceled)

43. (canceled)

44. (canceled)

45. (canceled)

46. (canceled)

47. (canceled)

48. The display system of claim 1, wherein two or more of or each of the at least one distinct peptides or two or more of or each of the library of peptides are concatenated.

49. (canceled)

50. The display system of claim 48, wherein each of the at least one distinct peptides is concatenated in a single polypeptide.

51. (canceled)

52. (canceled)

53. (canceled)

54. The display system of claim 1, wherein the binding motifs are selected from IgG isotype specific binding motifs, IgM isotype specific binding motifs, or a combination of IgG and IgM isotype specific binding motifs.

55. (canceled)

56. A method of determining the presence of specimen antibodies specific for Leptospirosis in a biological sample comprising:
contacting the display system of claim 1 with a biological sample, wherein the biological sample comprises a plurality of antibodies, wherein the plurality of antibodies is known or suspected to comprise specimen antibodies specific for Leptospirosis, and wherein the contacting is under conditions sufficient for the specimen antibodies specific for Leptospirosis to bind a cognate epitope;
measuring the binding between the at least one distinct peptide and the specimen antibodies; and
determining specimen antibodies specific for Leptospirosis are present in the biological sample when binding between at least one of the at least one distinct peptides and the specimen antibodies is detected.

57-111. (canceled)

112. A peptide expression library, wherein the library comprises a library of nucleic acid sequences encoding a library of peptides, wherein the library of peptides comprises at least one distinct peptide, wherein the at least one distinct peptide comprises a binding motif, wherein the binding motif is selected from a motif group consisting of: [FM]TEX[FY]N (SEQ ID NO:4), KXGHDXC (SEQ ID NO:5), KGGXDX[IV] (SEQ ID NO:6), KXHG[IV]F (SEQ ID NO:7), HWFDXW (SEQ ID NO:8), HW[FL]DX[WF] (SEQ ID NO:9), ILQAD (SEQ ID NO: 10), LHADXXF (SEQ ID NO:11), [IVTP]LHAD (SEQ ID NO:12), TLXAD[RSQ] (SEQ ID NO:13), KXIPAE (SEQ ID NO:14), [ED]XAGYN (SEQ ID NO: 15), [EDPA]X[AG][GA]YN (SEQ ID NO: 16), C[RL]XTDC (SEQ ID NO:17), [LIV]L[QH]AE (SEQ ID NO:18), LPADR (SEQ ID NO:19), [ML]XXLSAD (SEQ ID NO:20), [FWY]XP[RV]AD (SEQ ID NO:21), AD[QR]E[HQ] (SEQ ID NO:22), [FYL]XSAQP (SEQ ID NO:23), [SD]AQPX[WF] (SEQ ID NO:24), [TS]AQPXX[WR] (SEQ ID NO:25), MAGM[ED] (SEQ ID NO:26), CMAGM (SEQ ID NO:27), CMAGE (SEQ ID NO:28), GPAG[EQ] (SEQ ID NO:29), PAAG[TD] (SEQ ID NO:30), PAG[TD]XXX[WRSHTG] (SEQ ID NO:31), PA[GA][TD]XW (SEQ ID NO:32), [AP]AGT[MLI] (SEQ ID NO:33), CXDPQC (SEQ ID NO:34), [LF]EDKS (SEQ ID NO:35), and SXNLEQ (SEQ ID NO:36).

113-236. (canceled)
